(19) United States
(12) Patent Application Publication
(10) Pub. No.: US 2026/0306800 A1
(43) Pub. Date: Oct. 8, 2026

(54) SECUREMENT DEVICES, ASSEMBLIES, AND KITS FOR VASCULAR ACCESS DEVICES AND ASSOCIATED EXTENSION SETS

(71) Applicant: BECTON, DICKINSON AND COMPANY, Franklin Lakes, NJ (US)

(72) Inventors: Manish Kumar, Bengaluru (IN); Praveen Nalawade, Bengaluru (IN); Karthik MR, Bangalore (IN); Shishir Prasad, Ramsey, NJ (US)

(21) Appl. No.: 19/481,322

(22) PCT Filed: May 30, 2023
(86) PCT No.: PCT/US2023/023834

§ 371 Date: Nov. 4, 2025

(51) Int. Cl. A61M 25/02

(52) U.S. Cl. CPC A61M 25/02; A61M 2025/0266; A61M 2025/028

(57) ABSTRACT

A securement device configured to be connected with multiple extension tubes extending from an adapter of a multi-lumen vascular access device includes at least one base having a first surface with an adhesive configured for securing the at least one base to a patient and an opposing outwardly facing second surface. The securement device also includes at least one lumen receptor. The at least one lumen receptor is connected to and extends from the second surface of the at least one base. The at least one lumen receptor is configured to removably receive one or more of the multiple extension tubes of the multi-lumen vascular access device for securing the multiple extension tubes of the multi-lumen vascular access device to the at least one base.

### BACKGROUND OF THE INVENTION

### Field of the Invention

The present disclosure relates generally to securement or anchoring devices for stabilizing portions of a catheter or vascular access device against skin of a patient and, in particular, to securement devices and assemblies for securing multiple components of a multi-lumen vascular access device (e.g., a catheter hub, adapter, extension tubes, additional extension sets, etc.) to the patient's skin at desired positions, as well as to assemblies and kits including multiple securement devices and vascular access device components.

### Description of Related Art

A vascular access device, such as a catheter set and/or infusion set, is commonly used to infuse fluids into vasculature of a patient. For example, the vascular access device may be used for infusing normal saline solution, various medicaments, or total parenteral nutrition. The vascular access device may also be used for withdrawing blood from the patient. However, infusion of saline solution, various medicaments, or total parenteral nutrition is stopped prior to withdrawing the blood from the patient and collecting a blood sample. Therefore, there may be a delay in delivering needed fluids to the patient or a clinician may access the vasculature of the patient with an additional needle stick, which may result in harm to the patient.

Repeated access to the vascular access device to withdraw blood may increase a risk of introducing microbes into the vascular access device and infection. Repeated access to the vascular access device to withdraw blood may also increase a risk of thrombus and occlusion within a lumen of the vascular access device due to blood flowing through the vascular access device without thorough and proper flushing of the vascular access device.

In order to reduce the delay in delivering fluids to the patient and/or to avoid the need for multiple needle sticks or using the same line for fluid delivery and blood withdrawal, multi-lumen vascular access devices are available including a lumen for fluid delivery and a second lumen for another use, such as withdrawing blood. However, multi-lumen vascular access devices often include many components including a catheter hub or adapter, multiple extension tubes, and/or additional extension tubes and extension sets. The large number of separable components can be difficult to secure to the patient in an organized manner meaning that some components, such as extension tubes and/or additional extension sets, dangle freely and can become tangled, twisted, or kinked. Therefore, there is a need for securement or anchoring assemblies and devices for securing components of a multi-lumen vascular access device to a patient in an organized manner, as provided by the current disclosure.

### SUMMARY OF THE INVENTION

According to an aspect of the disclosure, a securement device configured to be connected with multiple extension tubes extending from an adapter of a multi-lumen vascular access device includes at least one base having a first surface with an adhesive configured for securing the at least one base to a patient and an opposing outwardly facing second surface and at least one lumen receptor that is connected to and extends from the second surface of the at least one base. The at least one lumen receptor is configured to removably receive one or more of the multiple extension tubes of the multi-lumen vascular access device for securing the multiple extension tubes of the multi-lumen vascular access device to the at least one base.

According to another aspect of the disclosure, a securement assembly for securement of multiple extension tubes extending from an adapter of a multi-lumen vascular access device includes at least one adapter securement device having an adhesive base and an adapter receptor connected to the base configured to removably engage the adapter of the multi-lumen vascular access device. The securement assembly also includes at least one lumen securement device having an adhesive base configured to removably receive the multiple extension tubes of the multi-lumen vascular access device for securing the multiple extension tubes of the multi-lumen vascular access device to the adhesive base of the at least one lumen securement device.

According to another aspect of the disclosure, a securement assembly for securement of multiple extension tubes extending from an adapter of a multi-lumen vascular access device includes at least one adapter securement device having an adhesive base and an adapter receptor connected to the base configured to removably engage the adapter of the multi-lumen vascular access device. The assembly also includes any of the previously described securement devices configured to engage one or more of the multiple extension tubes of the multi-lumen vascular access device.

According to another aspect of the disclosure, a kit for securing a multi-lumen vascular access device to a patient includes a multi-lumen vascular access device having a multi-lumen catheter including a distal end configured for insertion to vasculature of a patient, an adapter connected to a proximal end of the multi-lumen catheter, multiple extension tubes extending proximally from the adapter, and connectors at proximal ends of the extension tubes. The kit also includes at least one adapter securement device comprising an adhesive base and an adapter receptor connected to the base configured to removably engage the adapter of the multi-lumen vascular access device, and at least one lumen securement device. The at least one lumen securement device includes an adhesive base. The at least one lumen securement device is configured to removably receive the multiple extension tubes of the multi-lumen vascular access device for securing the extension tubes of the multi-lumen vascular access device to the adhesive base of the at least one lumen securement device.

According to another aspect of the disclosure, a method for securing extension tubes of a multi-lumen vascular access device to a patient includes attaching an adapter securement device to skin of a patient. The adapter securement device includes an adhesive base and an adapter receptor connected to the base configured to removably engage an adapter of the multi-lumen vascular access device. The method further includes steps of mounting the adapter of the multi-lumen vascular access device to the adapter receptor; attaching a lumen securement device to the patient at a position proximate to but spaced apart from the adapter securement device. The lumen securement device includes at least one lumen receptor connected to and extending from a base of the lumen securement device. The method further includes attaching one of the extension tubes of the multi-lumen vascular access device to the at least one lumen receptor.

Non-limiting illustrative examples of embodiments of the present disclosure will now be described in the following numbered clauses.

Clause 1: A securement device configured to be connected with multiple extension tubes extending from an adapter of a multi-lumen vascular access device, the securement device comprising: at least one base comprising a first surface comprising an adhesive configured for securing the at least one base to a patient and an opposing outwardly facing second surface; and at least one lumen receptor connected to and extending from the second surface of the at least one base, wherein the at least one lumen receptor is configured to removably receive one or more of the multiple extension tubes of the multi-lumen vascular access device for securing the multiple extension tubes of the multi-lumen vascular access device to the at least one base.

Clause 2: The securement device of clause 1, wherein the at least one lumen receptor comprises a plurality of lumen receptors, each of which is configured to removably receive one of the multiple extension tubes of the multi-lumen vascular access device for securing the multiple extension tubes to the at least one base.

Clause 3: The securement device of clause 2, wherein the at least one base is an elongated shape having a central longitudinal axis, and wherein the plurality of lumen receptors are aligned along a virtual line parallel or substantially parallel with the longitudinal axis of the at least one base.

Clause 4: The securement device of any of clauses 1-3, wherein the at least one lumen receptor is configured to removably receive connectors of the multiple extension tubes of the multi-lumen vascular access device.

Clause 5: The securement device of any of clauses 1-4, wherein the at least one base comprises an anchor pad comprising a breathable polyester material.

Clause 6: The securement device of clause 1, wherein the at least one base comprises an absorbent hydrocolloid material.

Clause 7: The securement device of any of clauses 1-6, wherein the at least one lumen receptor comprises a first support and a second support connected to the second surface of the at least one base and a cradle connected between the first support and the second support configured to removably receive a connector of one of the extension tubes of the multi-lumen vascular access device.

Clause 8: The securement device of clause 7, wherein the cradle is sized to provide a removable snap fit engagement with an outer surface of the luer connector of the extension tube of the multi-lumen vascular access device.

Clause 9: The securement device of clause 7 or clause 8, wherein the cradle is pivotally connected to the first support and the second support.

Clause 10: The securement device of any of clauses 1-9, wherein the at least one lumen receptor comprises an arcuate receiving portion sized to form a snap fit engagement with a distal end of a connector of one of the multiple extension tubes of the multi-lumen vascular access device.

Clause 11: The securement device of any of clauses 1-10, further comprising an adapter receptor connected to the second surface of the at least one base configured to removably engage the adapter of the multi-lumen vascular access device.

Clause 12: The securement device of clause 11, wherein the adapter receptor comprises a body defining a cavity sized to receive the adapter, a plurality of protrusions sized to be inserted through corresponding eyelets of the adapter, and a cover configured to be positioned over the protrusions and/or adapter for removably securing the adapter to the adapter receptor.

Clause 13: The securement device of clause 11 or clause 12, wherein the at least one base comprises an adapter securement portion connected to the adapter receptor, a lumen securement portion connected to the plurality of lumen receptors, and an intermediate portion between the adapter securement portion and the lumen securement portion.

Clause 14: The securement device of clause 13, wherein the adapter securement portion and the lumen securement portion are elongate shapes each heaving a longitudinal axis and the intermediate portion is an elongate shape with a longitudinal axis that is transverse or substantially transverse to the longitudinal axes of the adapter securement portion and the lumen securement portion.

Clause 15: The securement device of any of clauses 1-14, wherein the at least one lumen receptor comprises a support comprising a bottom connected to the at least one base, a top opposite the bottom, and an annular side surface extending between the top and the bottom, the support being rotatably connected to the at least one base and defining a channel sized to receive a section of one of the extension tubes of multi-lumen vascular access device.

Clause 16: The securement device of clause 15, wherein the channel curves about a central longitudinal axis of the support.

Clause 17: The securement device of clause 15 or clause 16, wherein the channel extends inwardly from the top of the support, such that the section of the extension tube is configured to be pressed into the channel through the top of the support.

Clause 18: The securement device of any of clauses 1-17, wherein the at least one lumen receptor comprises at least one strap comprising ends connected to the at least one base for securing the multiple extension tubes to the at least one base.

Clause 19: The securement device of clause 18, wherein the ends of the at least one strap are connected to the at least one base by portions of a hook and loop fastener fabric on the at least one strap and/or on the at least one base.

Clause 20: The securement device of any of clauses 1-19, wherein the at least one lumen receptor comprises depressions or holes sized to receive corresponding pins extending from a connector of one of the extension tubes of the multi-lumen vascular access device for pivotally connecting the connector to the at least one lumen receptor.

Clause 21: The securement device of clause 20, wherein the at least one lumen receptor comprises a first support and a second support positioned to allow the connector to be inserted between the first and second supports, and wherein each of the supports comprises one of the depressions or holes positioned to receive corresponding pins of the connector.

Clause 22: The securement device of any of clauses 1-21, wherein the at least one lumen receptor comprises four lumen receptors, with two of the lumen receptors configured to receive the multiple extension tubes and two of the lumen receptors configured to receive additional extension tubes of an extension set.

Clause 23: The securement device of clause 22, wherein the four lumen receptors are aligned along a virtual line that is parallel with or substantially parallel with a longitudinal axis of the at least one base.

Clause 24: A securement assembly for securement of multiple extension tubes extending from an adapter of a multi-lumen vascular access device, the securement assembly comprising: at least one adapter securement device comprising an adhesive base and an adapter receptor connected to the base configured to removably engage the adapter of the multi-lumen vascular access device; and at least one lumen securement device comprising an adhesive base configured to removably receive the multiple extension tubes of the multi-lumen vascular access device for securing the multiple extension tubes of the multi-lumen vascular access device to the adhesive base of the at least one lumen securement device.

Clause 25: The securement assembly of clause 24, wherein the lumen securement device comprises a plurality of lumen receptors, each of which is configured to removably receive one of the multiple extension tubes of the multi-lumen vascular access device for securing the multiple extension tubes to the adhesive base of the lumen securement device.

Clause 26: The securement assembly of clause 25, wherein each of the plurality of lumen receptors comprises a first support and a second support connected to the adhesive base and a cradle connected between the first support and the second support configured to removably receive a connector of one of the extension tubes of the multi-lumen vascular access device.

Clause 27: The securement assembly of clause 26, wherein the cradle is pivotally connected to the first support and the second support.

Clause 28: The securement assembly of any of clauses 25-27, wherein the plurality of lumen receptors each comprise an arcuate receiving portion sized to form a snap fit engagement with a distal end of a connector of one of the extension tubes of the multi-lumen vascular access device.

Clause 29: The securement assembly of any of clauses 25-27, wherein the plurality of lumen receptors comprises at least one additional lumen receptor configured to receive a connector of an additional extension tube connected to one of the extension tubes of the multi-lumen vascular access device.

Clause 30: The securement assembly of any of clauses 24-29, wherein the at least one lumen securement device comprises reusable tape.

Clause 31: The securement assembly of any of clauses 24-30, further comprising the multi-lumen vascular access device comprising the adapter and the multiple extension tubes extending from the adapter and comprising connectors at proximal ends thereof.

Clause 32: The securement assembly of clause 31, further comprising at least one additional extension tube comprising a distal end connected to the connector of one of the multiple extension tubes and a proximal end connected to a lumen receptor of the at least one lumen securement device.

Clause 33: The securement assembly of clause 31 or clause 32, wherein one or more of the multiple extension tubes of the multi-lumen vascular access device are pivotally or rotatably engaged to the at least one lumen securement device.

Clause 34: A securement assembly for securement of multiple extension tubes extending from an adapter of a multi-lumen vascular access device, the securement assembly comprising: at least one adapter securement device comprising an adhesive base and an adapter receptor connected to the base configured to removably engage the adapter of the multi-lumen vascular access device; and the securement device of any of clauses 1-23 configured to engage one or more of the multiple extension tubes of the multi-lumen vascular access device.

Clause 35: A kit for securing a multi-lumen vascular access device to a patient, comprising: a multi-lumen vascular access device comprising a multi-lumen catheter comprising a distal end configured for insertion to vasculature of a patient, an adapter connected to a proximal end of the multi-lumen catheter, multiple extension tubes extending proximally from the adapter, and connectors at proximal ends of the extension tubes; at least one adapter securement device comprising an adhesive base and an adapter receptor connected to the base configured to removably engage the adapter of the multi-lumen vascular access device; and at least one lumen securement device comprising an adhesive base, wherein the at least one lumen securement device is configured to removably receive the multiple extension tubes of the multi-lumen vascular access device for securing the extension tubes of the multi-lumen vascular access device to the adhesive base of the at least one lumen securement device.

Clause 36: The kit of clause 35, wherein the at least one lumen securement device comprises reusable tape.

Clause 37: The kit of clause 35 or clause 36, further comprising an extension set comprising at least one additional extension tube configured to be connected to one of the connectors on the proximal end of the extension tube.

Clause 38: The kit of clause 37, wherein the at least one lumen securement device comprises a plurality of lumen receptors, wherein at least two of the plurality of lumen receptors are configured to engage the connectors on the proximal ends of the extension tubes and at least two of the plurality of lumen receptors are configured to engage proximal connectors of the at least one additional extension tube of the extension set.

Clause 39: The kit of any of clauses 35-38, wherein the multiple extension tubes comprise curved sections configured to be received within a curved channel of a lumen receptor.

Clause 40: The kit of any of clauses 35-38, wherein the adhesive base of the at least one adapter securement device and the adhesive base of the at least one lumen securement device are integrally formed.

Clause 41: A method for securing extension tubes of a multi-lumen vascular access device to a patient comprising: attaching an adapter securement device to skin of a patient, the adapter securement device comprising an adhesive base and an adapter receptor connected to the base configured to removably engage an adapter of the multi-lumen vascular access device; mounting the adapter of the multi-lumen vascular access device to the adapter receptor; attaching a lumen securement device to the patient at a position proximate to but spaced apart from the adapter securement device, wherein the lumen securement device comprises at least one lumen receptor connected to and extending from a base of the lumen securement device; and attaching one of the extension tubes of the multi-lumen vascular access device to the at least one lumen receptor.

Clause 42: The method of clause 41, wherein the lumen securement device comprises a plurality of lumen receptors, the method further comprising attaching each of the extension tubes to one of the plurality of lumen receptors.

Clause 43: The method of clause 41 or clause 42, further comprising attaching an additional extension tube to a connector on the proximal end of one of the extension tubes of the vascular access device and attaching a proximal connector of one of the additional extension tube to one of the plurality of lumen receptors of the lumen securement device.

Clause 44: The method of clause 43, wherein the proximal connector is attached to the lumen receptor in a distally facing direction with the additional extension tube in a J-loop configuration.

Clause 45: The method of clause 43 or clause 44, wherein the proximal connector is attached to the lumen receptor in a proximally facing direction with the additional extension tube in a U-loop configuration.

Clause 46: The method of any of clauses 41-45, further comprising pivoting a connector of the extension tube connected to the at least one lumen receptor relative to the base of the lumen securement device to improve accessibility of the connector.

Clause 47: The method of clause 46, further comprising, after pivoting the connector to improve accessibility of the connector, at least one of scrubbing, disinfecting, capping, or attaching a fluid delivery device to the connector.

### BRIEF DESCRIPTION OF THE DRAWINGS

FIG. 1A is a perspective view of a securement assembly for securing a multi-lumen vascular access device to a patient, according to an aspect of the present disclosure.

FIG. 1B is a perspective view of luer connectors and a lumen securement device of the assembly of FIG. 1A.

FIG. 1C is a schematic drawing showing steps for attaching components of the multi-lumen vascular access device to the securement assembly of FIG. 1A, according to an aspect of the disclosure.

FIG. 2A is a perspective view of another example of a securement assembly for securing a multi-lumen vascular access device to a patient, according to an aspect of the present disclosure.

FIG. 2B is another perspective view of the securement assembly of FIG. 2A, with one of the luer connectors tilted in an upward direction relative to the lumen securement device of the securement assembly.

FIG. 2C is a top view of the luer connectors connected to lumen receptors of the lumen securement device of FIG. 2B.

FIG. 3A is a perspective view of the lumen securement device of the assembly of FIG. 2A.

FIG. 3B is a perspective view of the luer connectors of the multi-lumen vascular access device of FIG. 2A.

FIG. 3C is an enlarged perspective view of one of the luer connectors of FIG. 3B.

FIG. 4A is a perspective view of another example of a securement assembly for components of a multi-lumen vascular access device, according to an aspect of the present disclosure.

FIG. 4B is a schematic drawing showing steps for attaching components of the multi-lumen vascular access device to the securement assembly of FIG. 4A.

FIG. 4C is a perspective view of a lumen securement device of the securement assembly of FIG. 4A.

FIG. 5A is a perspective view of another example of a securement assembly for securing a multi-lumen vascular access device to a patient, according to an aspect of the present disclosure.

FIG. 5B is a perspective view of a securement device of the assembly of FIG. 5A without the vascular access device.

FIG. 6A is a perspective view of another example of a securement assembly for securing a multi-lumen vascular access device to a patient, according to an aspect of the present disclosure.

FIG. 6B is a schematic drawing showing steps for attaching components of the multi-lumen vascular access device to the securement assembly of FIG. 6A, according to an aspect of the disclosure.

FIG. 7 is a perspective view of another example of a securement assembly for securing a multi-lumen vascular access device to a patient, according to an aspect of the present disclosure.

FIG. 8A is a perspective view of a kit of parts that can be assembled for securing a multi-lumen vascular access device to a patient, according to an aspect of the present disclosure.

FIGS. 8B and 8C are perspective views of re-sealable tape of the kit of FIG. 8A.

FIG. 9 is a perspective view of an example of a securement assembly for securing a multi-lumen vascular access device and additional extension tubes to a patient, according to an aspect of the present disclosure.

FIG. 10 is a perspective view of another example of a securement assembly for securing a multi-lumen vascular access device and additional extension tubes to a patient, according to an aspect of the present disclosure.

FIG. 11 is a perspective view of another example of a securement assembly for securing a multi-lumen vascular access device and additional extension tubes to a patient, according to an aspect of the present disclosure.

FIG. 12A is a perspective view of a multi-lumen vascular access device that can be used with the securement devices and assemblies disclosed herein, according to an aspect of the present disclosure.

FIG. 12B is a cross-sectional view of the multi-lumen vascular access device of FIG. 12A.

FIG. 12C is a perspective view showing a fluid delivery device connected to the multi-lumen vascular access device of FIG. 12A, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. As used herein, the term “proximal” refers to a portion or end of a device, such as a syringe or catheter, which is grasped, manipulated, or used by a practitioner or another user. The term “distal” refers to an end or portion of the device that is farthest away from the portion of the device that is grasped, manipulated, or used by the practitioner. For example, the “proximal end” of a catheter or intravenous (IV) line refers to the end including a fluid port that is connected to a fluid container, such as an IV bag or syringe. The “distal end” of the catheter or IV line refers to the end that is connected to the patient. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to securement assemblies 110, devices 112, 114, and kits for securing components of a vascular access device, such as a multi-lumen vascular access device 10 or catheter set to a patient. For example, the assemblies 110, devices 112, 114, and kits can include adhesive members, such as pads, bandages, tape, or similar adhesive materials for attaching components of the vascular access device 10 to skin and/or clothing of a patient.

The securement assemblies 110, devices 112, 114, and kits of the present disclosure are provided for stabilizing portions of a catheter or vascular access device against the skin of a patient. In particular, the assemblies 110, devices 112, 114, and kits disclosed herein are configured to quickly and easily secure components of the vascular access device for extended periods of time to prevent axial displacement of the vascular access device, such as displacement caused by tugging on external extensions of the catheter. By contrast, catheter components are conventionally secured to a patient by one of: suturing a catheter hub to a patient's skin through eyelets in wings extending from the catheter hub; applying an adhesive over the catheter hub thereby securing the hub to the patient's skin; and/or placing the hub in a semi-flexible securement device which is held to the patient's skin by an adhesive base. However, any of these securement methods can be time consuming and/or difficult to implement. Further, such conventional securement methods do not secure long-hanging or dangling catheter extensions, such as additional extension tubes of an extension set. As such, using conventional securing methods, long-hanging catheter extensions can become tangled in clothing or other articles, which can cause axial movement of components of the catheter set. Such axial movement can introduce pathogens along extra-luminal surfaces through a skin insertion site. Also, axial movement can cause catheter extensions and accessories (e.g., caps, clamps, etc.) to break, become un-snapped, or kink, which can compromise an entire catheter set presenting a risk to patient safety.

In view of such difficulties with conventional securement methods, the securement assemblies 110, devices 112, 114, and kits disclosed herein are configured to provide a connecting location within close proximity to a vascular access site and allow for attachment of various extension tubes and similar accessories. As such, the securement assemblies 110, devices 112, 114, and kits can reduce a total surface of an extravascular portion of a vascular access device, which can be a predominant site for contamination, facilitate improved placement of insertion-site dressing, and reduce a potential for snagging irritation, and other problems that can be associated with dangling catheter extensions.

The securement assemblies 110 and devices 112, 114 disclosed herein can be used by a healthcare workers or clinician to ensure that portions of multi-lumen vascular access device and/or extension sets connected thereto are securely held in place against the paint and do not dangle or twist in an unsafe manner. As used herein, the “healthcare worker” or clinician can be a medical professional, such as a medical technician or nurse, trained to perform a medical procedure, such as a fluid delivery or blood collection procedure.

The securement assemblies 110, devices 112, 114, and kits of the present disclosure can be configured to securely attach a variety of catheters, intravascular access devices, and similar medical devices in place against the patient. Many different arrangements of vascular access devices are commercially available from different manufactures. Exemplary multi-lumen vascular access devices 10 that can be used with the securement devices 112, 114 and assemblies 110 disclosed herein are described in U.S. Patent Application Publication No. 2021/0290898A1 entitled “Multi-lumen vascular access device and related methods” which is incorporated herein by reference in its entirety.

As described in further detail herein, a multi-lumen vascular access device 10, which can be used with the securement assemblies 110, devices 112, 114, and kits disclosed herein, generally includes a catheter 40 and/or other fluid delivery components, such as extension tubes 36, 38, for delivering fluids into vascular of a patient through separate lumens 12, 18. For example, a first medical fluid can be delivered or withdrawn through the first lumen 12 and another medical fluid can be delivered or withdrawn through the second lumen 18. The multi-lumen vascular access device 10 generally comprises a multi-lumen tube, such as a proximal portion 48 of the catheter 40. The catheter 40 also includes a distal end 42 configured for insertion to vasculature of a patient at a vascular access site. The proximal portion 48 of the multi-lumen tube is connected to an adapter 30 or hub. The multi-lumen vascular access device 10 also includes the multiple single-lumen extension tubes 36, 38 extending from a proximal end 34 of the adapter 30. The extension tubes 36, 38 can have connectors 26, 28, such as male or female luer connectors, threaded connectors, or snap-fit connectors, attached to a proximal end 16, 22 of each lumen 12, 18 for connecting the lumens 12, 18 to fluid sources, fluid delivery devices, and/or fluid containers or sample collection devices. In particular, the connectors 26, 28 can be configured to engage a fluid delivery device, such as a syringe, or catheter introducer for delivery fluids and/or other objects through the extension tubes and into the lumens 12, 18 of the multi-lumen vascular access device 10.

As previously described, the connectors 26, 28 can be male or female luer connectors. As used herein, a “luer connector” refers to a connector that includes a tapered portion (i.e., a luer taper) for creating a friction engagement between a distal tip of a male connector and a tapered cavity of the female connector configured to receive and engage the distal tip. In particular, a distal tip of a male luer connector can be configured to be used with female needleless connectors, specifically female luer connectors for a range of dimensions permitted by various design protocols, such as ISO 80369-7:2016 or ISO 80369-7:2021 (Female Luer Lock Connector dimensions).

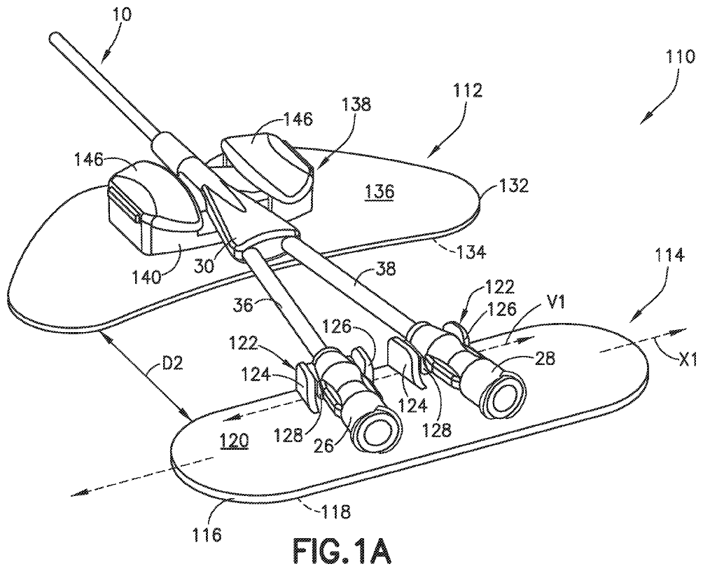

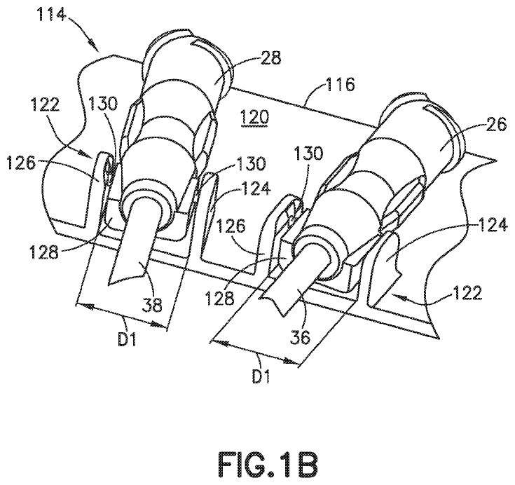

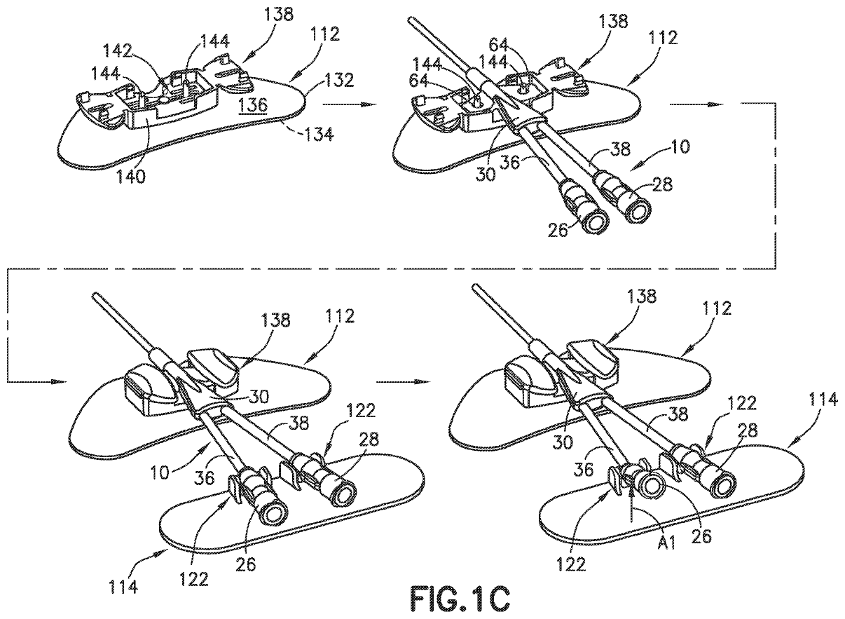

FIGS. 1A-1C show a securement assembly 110 for connecting the components of the multi-lumen vascular access device 10 in place against skin of the patient. The assembly 110 includes a catheter adapter securement device 112 that receives the adapter 30 of the vascular access device 10. The assembly 110 also includes one or more lumen securement devices 114 that receive portions of the lumens 12, 18, such as the extension tubes 36, 28 and/or connectors 26, 28 of the vascular access device 10, for securing the lumens 12, 18 to the patient.

As shown in FIG. 1A, the lumen securement device 114 comprises a base 116 comprising a first or bottom surface 118 comprising an adhesive configured for securing the base 116 to the patient and an opposing outwardly facing second or top surface 120. The base 116 can comprise, for example, polyethylene foam, a polyethylene liner, a synthetic rubber pad, polyethylene coated kraft tape, poly-coated kraft tape, white tricot knit fabric, nylon, TYVEK®, polyurethane film, cotton/polyimide fabric, and/or similar materials. The adhesive can be, for example, a hydrocolloid and/or zinc oxide material. The adhesive can also comprise a pressure sensitive adhesive, an acrylic adhesive, ethyl cyanoacrylate, and/or cyclohexane.

The adhesive can be a removable and/or reusable adhesive that secures the base 116 in place against, for example, skin or clothing of the patient and that can be easily removed without causing pain or damaging the base 116. In some examples, the base 116 can be an adhesive pad, such as a pad of the StatLock™ Dialysis Stabilization Device by Becton, Dickinson and Company of Franklin Lakes, N. J. For example, the base 116 can comprise an anchor pad comprising a breathable plastic sheet, such as a tricot polyester sheet. The sheet can be coated or covered with one or more absorbent layers, such as layers comprising an absorbent hydrocolloid.

In some examples, the base 116 can be an elongated shape, such as a rectangle or oval having a longitudinal axis X1. Dimensions of the base 116 can generally depend on where on the patient the base 116 will be adhered and/or on dimensions of components of the vascular access device 10. In some examples, the base 116 can be about 60 mm to about 100 mm in length and about 30 mm to about 40 mm in width. However, dimensions of the securement assembly 110 may be larger for large adult male patients and/or when used with vascular access devices 10 having more than two lumens and associated components. By contrast, dimensions of the base 116 may be smaller for devices intended for use with pediatric patients or small female adult patients.

The lumen securement device 114 further comprises one or multiple lumen receptors 122, also referred to as extension tube receptors or grippers, which are connected to and extends from the top surface 120 of the base 116. The lumen receptors 114 can be made from various rigid plastic materials, known in the art, and can be connected to the base 116 of the lumen securement device 114 using known adhesives or fasteners. For example, the lumen receptors 122 can comprise a rigid thermoplastic, such as at least one of polycarbonate, high density polyethylene (HDPE), polypropylene (PP), polyether ketone (PEEK), polytetrafluoroethylene (PTFE), acrylonitrile butadiene styrene (ABS), biaxially-oriented polyethylene terephthalate (BOPET), and/or polyimide.

The lumen receptors 122 are configured to removably receive portions of the multiple extension tubes 36, 38 and/or connectors 26, 28 of the multi-lumen vascular access device 10 for securing the extension tubes 36, 28 to the base 116. For example, the lumen receptor 122 can be configured to engage either a flexible tubular portion of the extension tubes 36, 38 or the more rigid connectors 26, 28 at the proximal end of the extension tubes 36, 28. In some examples, the lumen receptors 122 can be configured to allow the extension tubes 36, 28 and/or connectors 26, 28 to pivot or rotate relative to the base 116 so that a clinician can manipulate the connectors 26, 28 without first disconnecting the extension tubes 36, 38 from the lumen receptors 122. For example, the clinician may pivot a connector 26, 28 upwards in order to connect a fluid delivery device to the connector 26, 28 without first removing the connector 26, 28 from the lumen receptor 122. In a similar way, the clinician may pivot the connector 26, 28 upwards in order to scrub, disinfect, or cap the connector 26, 28 without first removing the connector 26, 28 from the lumen receptor 122.

In some examples, the lumen securement device 114 includes multiple separate lumen receptors 122, each of which is configured to receive one of the multiple extension tubes 36, 38 or connectors 26, 28 of the multi-lumen vascular access device 10 for securing the multiple extension tubes 36, 38 to the base 116. As shown in FIG. 1A, the multiple lumen receptors are aligned along a virtual line V1 that is parallel or substantially parallel (e.g., within about 10 degrees of parallel) with the longitudinal axis X1 of the base 116.

With continued reference to FIGS. 1A-1C, in some examples, each of the multiple lumen receptors 122 can include pairs of supports, such as first and second vertical plate or post, connected to the top surface 120 of the base 116. For example, the lumen receptors 112 can include a first support 124 and a second support 126 separated by a distance D1 (shown in FIG. 1B) sized to receive a portion of the extension tube 36, 38 or connector 26, 28. The distance D1 can be from about 30 mm to about 40 mm depending upon a size of the patient and/or components of the vascular access device 10.

The lumen receptor 122 also includes a cradle 128 connected between the first support 126 and the second support 128. The cradle 128 can define an arcuate or curved surface configured to removably receive the connector 26, 28 of one of the extension tubes 36, 38. In particular, the receiving surface of the cradle 128 can be shaped to create a snap fit engagement between the cradle 128 and an outer surface of one of the connectors 26, 28, as shown most clearly in FIG. 1B.

In some examples, the cradle 128 can be pivotally connected to the first support 124 and the second support 126. For example, the cradle 128 can include posts or pins 130 (shown in FIG. 1B) that are configured to be received within a depression, hole, or slot on inwardly facing surfaces of the first and second supports 124, 126 for creating a suitable hinged or pivoting engagement between the supports 124, 126 and the cradle 128. Advantageously, the hinged or pivoting engagement allows the clinician to pivot or move the connectors 26, 28 engaged to the cradle 128 upwardly away from the base 116. As previously described, when the connectors 26, 28 are pivoted upward away from the base 116, the clinician can more easily attach a fluid delivery device or introducer to the connector 26, 28. The clinician can also more easily scrub, disinfect, or cap the connector 26, 28 without needing to detach the connector from the lumen receptor.

With reference again to FIG. 1A, the securement assembly 110 further comprises the adapter securement device 112, also referred to as a catheter adapter or catheter hub securement device, for securing the adapter 30 of the multi-lumen vascular access device 10 to skin and/or clothing of the patient at a position spaced apart from the lumen securement device by a distance D2 (shown in FIG. 1A). The distance D2 can be about 50 mm to about 150 mm depending upon size of the patient and/or components of the vascular access device 10.

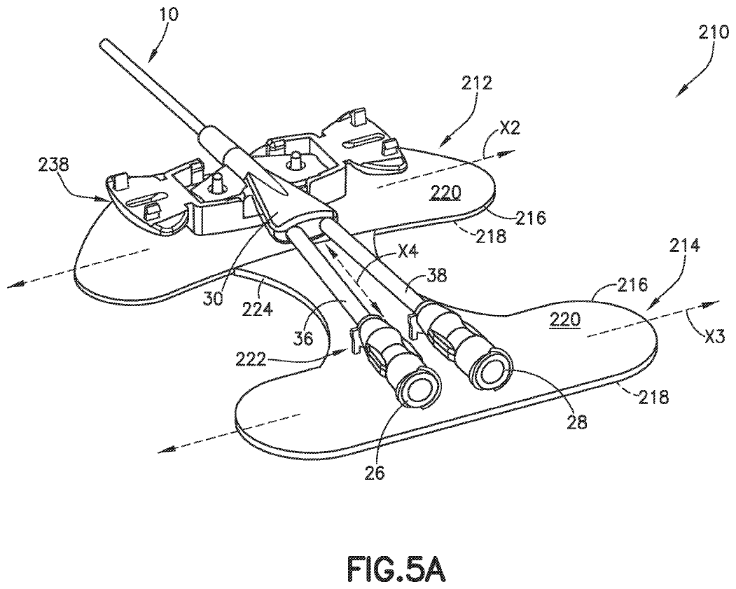

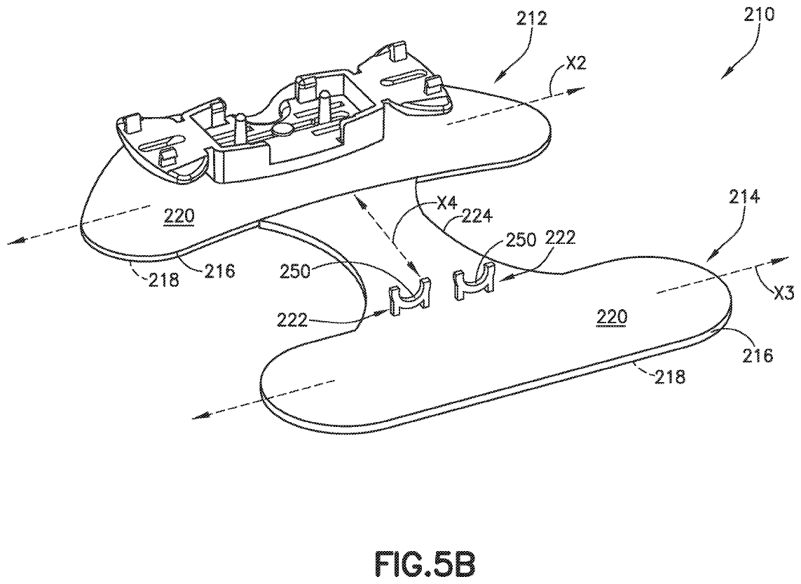

The adapter securement device 112 comprises a base 132, which can be similar in shape and structure to the base 116 of the lumen securement device 114. In some examples, as shown in FIGS. 5A and 5B, the base 116 of the lumen securement device 114 and the base 132 of the adapter securement device 112 can be integrally formed and/or connected together by an intermediate portion, connector, or fabric sheet. The base 132 of the adapter securement device 112 can include a first or bottom surface 134 comprising an adhesive for securing the device 112 to the skin and/or clothing of the patient and a second or top surface 136 opposite the bottom surface 134. The adapter securement device 112 also includes an adapter receptor 138. The adapter receptor 138 can be a rigid plastic structure connected to or extending from the base 132 of the adapter securement device 112. For example, the adapter receptor 138 can comprise a rigid thermoplastic, such as at least one of polycarbonate, high density polyethylene (HDPE), polypropylene (PP), polyether ketone (PEEK), polytetrafluoroethylene (PTFE), acrylonitrile butadiene styrene (ABS), biaxially-oriented polyethylene terephthalate (BOPET), and/or polyimide.

In some examples, the adapter receptor 138 comprises a housing or body 140 defining a cavity 142 sized to receive the adapter 30 of the multi-lumen vascular access device 10. As shown in FIG. 1C, the housing or body includes protrusions 144 extending into the cavity 142 sized to be inserted through corresponding eyelets 64 of the adapter 30. The adapter securement device 112 also includes a cover 146 (shown in FIGS. 1A and 1C) configured to be positioned over the cavity 142, protrusions 144, and adapter 30 for removably securing the adapter 30 to the adapter receptor 138.

FIG. 1C shows steps for connecting the multi-lumen vascular access device 10 to the securement assembly 110 shown in FIGS. 1A-1C. As shown in FIG. 1C, the adapter securement device 112 is provided. For example, the bottom surface 134 of the base 132 of the adapter securement device 112 can be adhered to skin or clothing of a patient at a position proximate to a vascular access site. Once the adapter securement device 112 is in place, the clinician inserts the adapter 30 of the multi-lumen vascular access device 10 into the cavity 142 defined by the housing or body 140 of the adapter receptor 138. Specifically, the eyelets 64 of the adapter 30 are inserted over the protrusions 144 extending into the cavity 142 of the body 140 for fixing the adapter 30 in place within the cavity 142. In order to secure the adapter 30 to the body 140, the cover 146 can also be attached to the body 140, thereby removably securing the adapter 30 to the adapter securement device 112. In a similar manner, the base 116 of the lumen securement device 115 is also adhered in place against the skin or clothing of the patient. Once the lumen securement device 144 is positioned at a desired location, the extension tubes 36, 38 are inserted into the lumen receptors 122. For example, as previously described, a connector 26, 28 of one of the extension tubes 36, 38 can be positioned on the cradle 128 of one of the lumen receptors 122, such that an outer surface of the connector 26, 28 contacts and forms a snap fit engagement with the receiving surface of the cradle 128. As shown in FIG. 1C, when the connectors 26, 28 are attached to the lumen receptors, the connectors 26, 28 are able to pivot, twist, or rotate in an upwards direction (shown by arrow Al in FIG. 1C) allowing the clinician to manipulate and/or attach other devices to the connectors 26, 28 without first removing the connectors 26, 28 from the lumen receptors 122 of the lumen securement device 114.

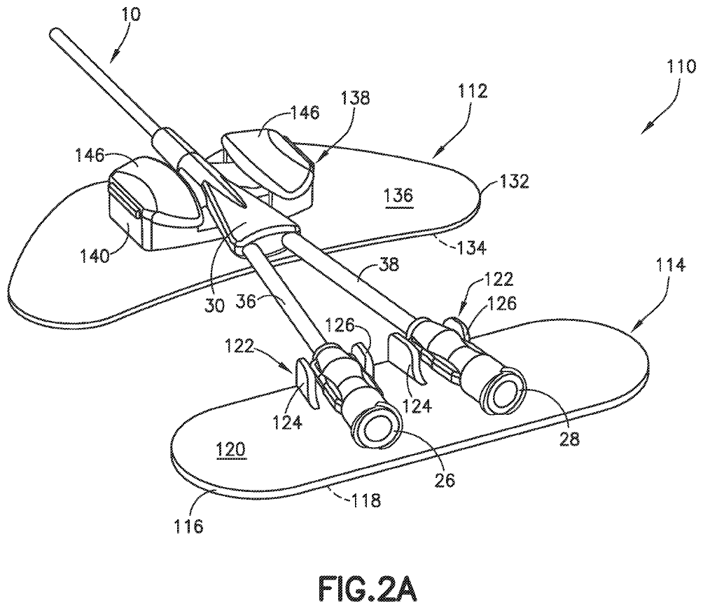

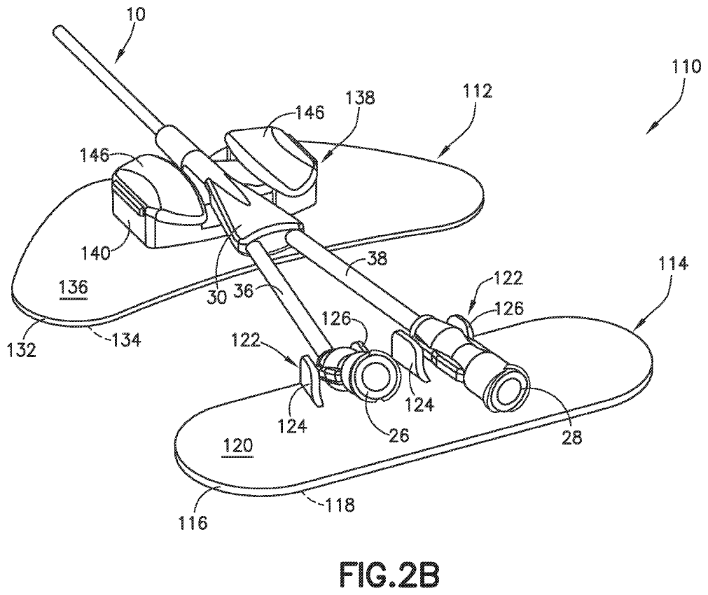

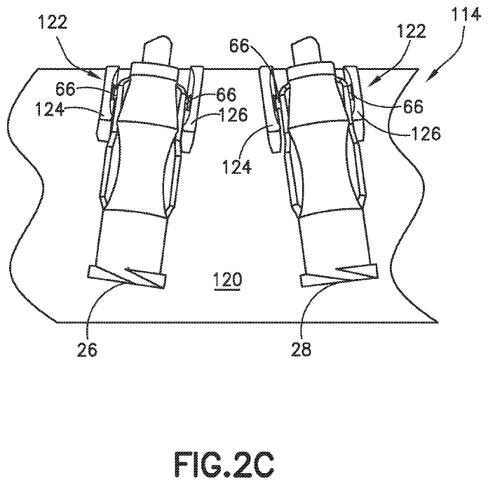

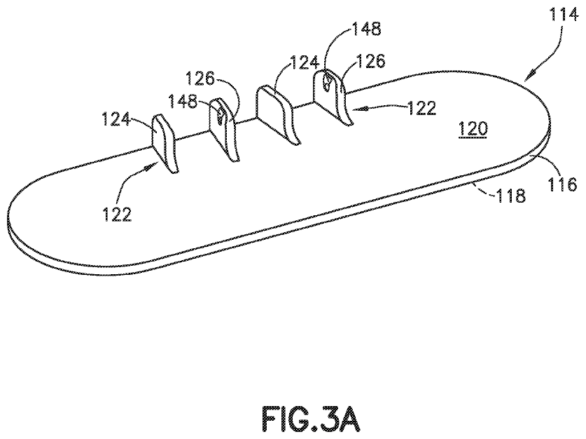

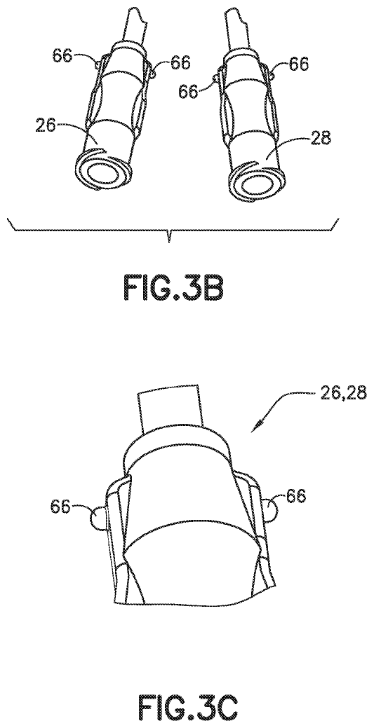

FIGS. 2A-3C show another example of a securement assembly 110 for securing the multi-lumen vascular access device 10 to the patient. The assembly 110 includes the anchor securement device 112 of previous examples and another example of a lumen securement device 114 for securing the extension tubes 36, 38 of the multi-lumen vascular access device 10 in place on skin and/or clothing of the patient.

As in previous examples, the lumen securement device 114 comprises the base 116 comprising the bottom surface 118 with an adhesive configured for securing the base 116 to the patient and the opposing outwardly facing top surface 120. The securement device also includes the multiple lumen receptors 122 that form a hinged engagement with connectors 26, 28 of the extension tubes 36, 28. However, unlike in previous examples, the lumen receptors 122 of FIGS. 2A-3C do not include a cradle 128 for receiving the connectors 26, 28. Instead, as shown in FIGS. 2A-3A, the lumen receptors 112 include the pairs of supports, such as the first support 124 and the second support 126 connected to the top surface 120 of the base 116. As shown most clearly in FIG. 3A, each support includes a depression 148, such as a hole or slot, on an inwardly facing surface thereof. The depression 148 is configured to receive a pin 66 (shown in FIGS. 2C, 3B, and 3C) extending outwardly from an outer surface of the connectors 26, 28. The connectors 26, 28 of the extension tubes 36, 38 can be inserted between the first and second supports 124, 126 of the lumen receptor 122 causing the pins 66 to snap into place within the depressions 148 or holes on the inwardly facing surfaces of the first and second supports 124, 126. Once the pins 66 are engaged with the depressions 148, the connectors 26, 28 can pivot moving upwardly relative to the base 116 as shown in FIG. 2B.

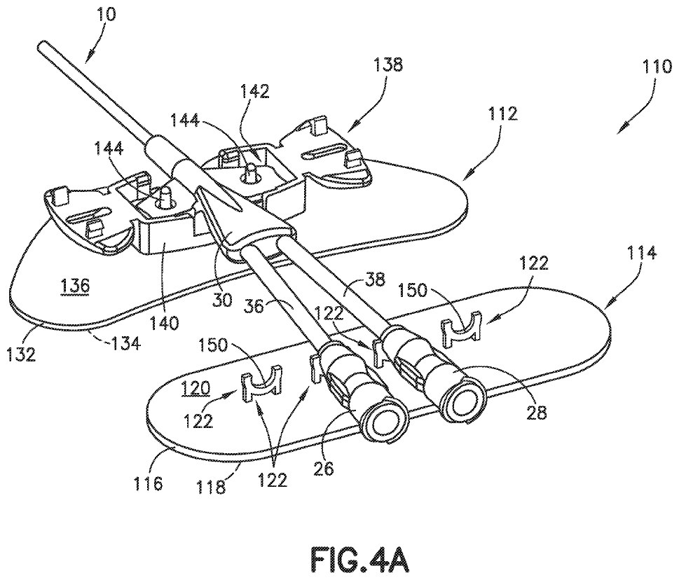

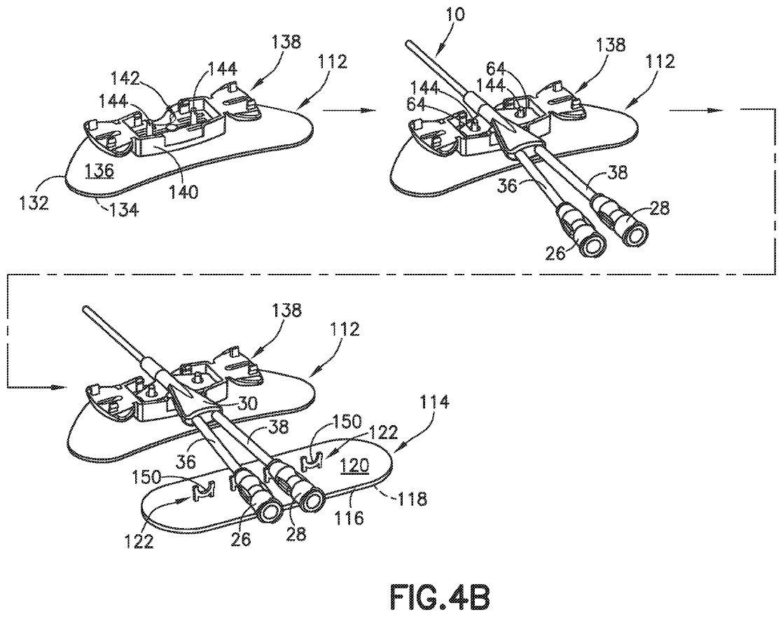

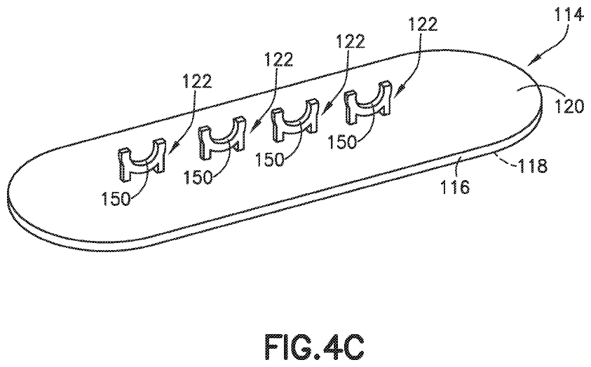

FIGS. 4A and 4B show another example of a securement assembly 110 for securing the multi-lumen vascular access device 10 to the patient. As in previous examples, the securement assembly 119 includes the adapter securement device 112 that secures the adapter 30 in place against skin or clothing of the patient proximate to a vascular access site. The securement assembly 110 also includes another example of a lumen securement device 114 (also shown in FIG. 4C) for securing portions of the extension tubes 36, 38 of the multi-lumen vascular access device 10 in place against skin and/or clothing of the patient.

As in previous examples, the lumen securement device 114 comprises the base 116 comprising the bottom surface 118 comprising an adhesive for securing the base 116 to the patient and an opposing outwardly facing top surface 120. The lumen securement device 114 also includes the lumen receptors 122, each of which is configured to removably receive one of the extension tubes 36, 38. Unlike in previous examples, the lumen receptors 122 of FIGS. 4A and 4B fixedly connect the extension tubes 36, 38 to the lumen securement device 114. For example, each lumen receptor 122 can include an arcuate receiving portion 150 sized to form a snap fit and fixed engagement with a narrow or distal end of the connectors 26, 28.

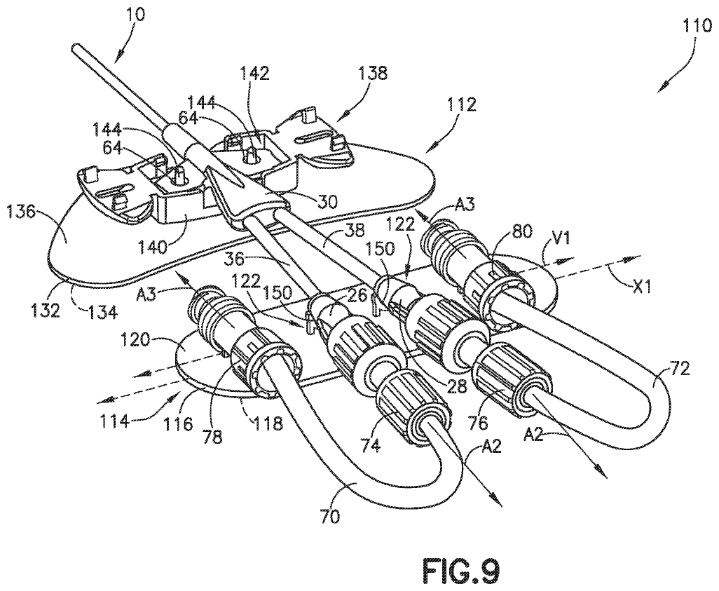

In some examples, the lumen securement device 114 includes multiple lumen receptors 122, such as two, three, four, or more lumen receptors 122. Including multiple lumen receptors means that the lumen securement device 114 can be used with multi-lumen vascular access devices 10 with multiple extension tubes (e.g., two, three, four or more extension tubes extending from the proximal end 34 of the adapter 30). Alternatively, or in addition, including more than two lumen receptors 122 means that the clinician can select which lumen receptor 122 is in a most convenient position for receiving a particular extension tube 36, 38 of the multi-lumen vascular access device 10. For example, in FIG. 4A, the extension tubes 36, 38 are connected to adjacent middle lumen receptors 122. However, in other situations and/or for other patients, it may be more convenient to attach the extension tubes 36, 38 to the outer lumen receptors 122 so that the connectors 26, 28 of the extension tubes 36, 38 are fixed farther apart from one another, which may make the connectors 26, 28 easier for the clinician to grasp and manipulate. Also, as described in detail in connection with FIGS. 9-11, the additional lumen receptors 122 can also be used for securing additional extension tubes, such as tubes of an extension set, and/or other tubular bodies to the lumen securement device 114 along with or in addition to the extension tubes 36, 38 of the multi-lumen vascular access device 10 shown in FIGS. 4A and 4B.

FIG. 4B shows steps for attaching portions of the multi-lumen vascular access device 10 to the securement assembly 110 of FIGS. 4A-4C. Initially, the clinician attaches the bottom surface 118 of the base 116 of the adapter securement device 112 to a convenient location on skin and/or clothing of the patient proximate to a vascular access site. The clinician then inserts the adapter 30 into the housing or body 140 of the adapter receptor 138, as in previous examples, such that protrusions 144 or posts of the body 140 are inserted through the eyelets 64 of the adapter 30. The clinician then adheres the lumen securement device 114 to the patient at a convenient location proximate to but spaced apart from the adapter securement device 112. Once the lumen securement device 114 is adhered to the patient, the clinician inserts one or more of the extension tubes 36, 38 into a corresponding lumen receptor 122 such that the connectors 26, 28 of the extension tubes 36, 38 contact and form a snap fit engagement with the receiving portion 150 of the lumen receptors 122. For example, as shown in FIGS. 4A and 4B, the narrow or distal ends of the connectors 26, 28 are inserted into the lumen receptors 122 contacting the receiving portions 150 of the lumen receptors 122 and forming a fixed engagement between the connectors 26, 28 and the lumen receptors 122.

FIGS. 5A and 5B show an example of an integrated securement device 210 for securing components of the multi-lumen vascular access device 10 to the patient. In particular, the securement device 210 is configured for securing the adapter 30 and multiple extension tubes 36, 38 and/or connectors 26, 28 to the patient with a single enlarged or integrated base 216.

As shown in FIGS. 5A and 5B, the enlarged or integral base 216 comprising a first or bottom surface 218 comprising an adhesive that securing the base to a patient and an opposing outwardly facing second or top surface 220. The securement device 210 also includes an adapter receptor 238 for securing the adaptor 30 of the multi-lumen vascular access device 10 to the base 216 and the multiple lumen receptors 222 for securing the extension tubes 36, 38 to the base 216. However, unlike in previous examples, the adapter receptor 238 and lumen receptors 222 are connected to different areas of a same enlarged pad or sheet. In particular, the base 216 includes an adapter securement portion 212 connected to the adapter 30, a lumen securement portion 214 connected to the lumen receptors 222, and a middle or intermediate portion 224 extending between the adapter securement portion 212 and the lumen securement portion 214.

In some examples, the adapter securement portion 212 and the lumen securement portion 214 are elongate shapes, each having a central longitudinal axis X2, X3. The intermediate portion 224 can also be an elongate shape with a longitudinal axis X4 that is transverse or substantially transverse (e.g., within about 10 degrees of transverse) to the longitudinal axes X2, X3 of the adapter securement portion 212 and the lumen securement portion 214.

As shown in FIGS. 5A and 5B, the lumen receptors 222 can be configured to fixedly engage the connectors 26, 28 of the extension tubes 36, 38 similar to the lumen receptors 122 of FIGS. 4A-4C described previously. In particular, as in the previous examples, the lumen receptors 222 can comprise the arcuate receiving surface or receiving portion 250 configured to contact and form a snap fit and fixed engagement with narrow or distal ends of the connectors 26, 28. In other examples, the enlarged or integral base 216 of FIGS. 5A and 5B can be used with any of the other exemplary lumen receptors 122 of the present disclosure. For example, the enlarged or integral base 216 of FIGS. 5A and 5B can be modified to include lumen receptors 222 that allow the connectors 26, 28 to pivot, twist, or rotate relative to the base 216 so that the clinician can manipulate the connectors 26, 28 without detaching the connectors 26, 28 from the lumen receptors 222. Also, the enlarged or integral base 216 can be modified to include additional (e.g., more than two) lumen receptors 222 so that the securement device 210 can be used with multi-lumen vascular access devices 10 having more than two extension tubes 36, 38 and/or to provide additional options for locations for attaching the extension tubes 36, 38 to the securement device 210. Also, the additional lumen receptors 222 can be used for securing additional extension tubes or catheter sets to the securement device 210.

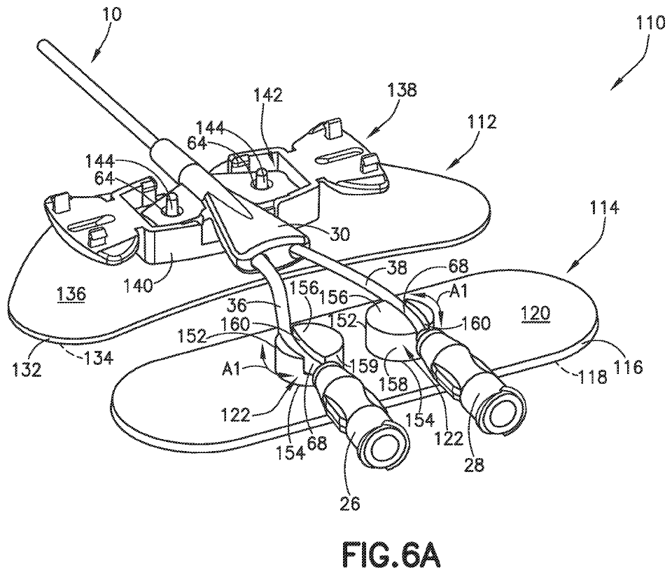

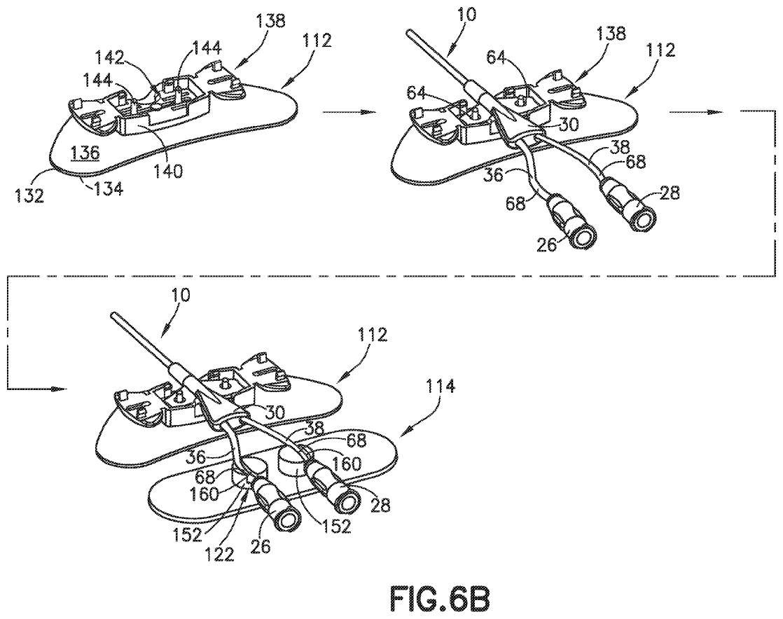

FIGS. 6A and 6B show another example of a securement assembly 110 for securing the multi-lumen vascular access device 10 to the patient. As in previous examples, the securement assembly 110 includes the adapter securement device 112 that secures the adapter 30 of the multi-lumen vascular access device 10 in place against skin or clothing of the patient proximate to a vascular access site. The assembly 110 also includes an example of a lumen securement device 114 for securing portions of the extension tubes 36, 38 of the multi-lumen vascular access device 10 in place against skin and/or clothing of the patient. Unlike in previous examples, the lumen receptors 122 of FIGS. 6A and 6B are configured to rotate relative to the base 116 allowing the clinician to rotate the extension tubes 36, 38 and/or connectors 26, 28 to more convenient positions in order to manipulate (e.g., scrub, disinfect, cap, and/or attach fluid delivery devices to) the connectors 26, 28.

As shown in FIGS. 6A and 6B, the lumen receptors 122 each include a support 152, such as a cylindrical post or body, comprising a bottom 154 connected to the base 116, a top 156 opposite the bottom 154, and an annular side surface 158 extending between the bottom 154 and the top 156. The support 152 is rotatably connected to the base 116 meaning that the clinician can rotate the support 152 and extension tube 36, 38 mounted thereto as shown by arrow Al (in FIG. 6A).

In some examples, the support 152 of the lumen receptor 122 defines a channel 160 sized to receive a portion of one of the extension tubes 36, 38 of multi-lumen vascular access device 10. In order to securely retain the extension tube within the channel 160, the channel 160 can be curved (e.g., curved about a longitudinal axis of the support 152) and configured to receive a corresponding curved section 68 of the extension tube 36, 38 for securing the extension tube 36, 38 to the support 152. In other examples, the channel 160 can include tapered or sloped walls, protrusions, or similar structures for retaining the curved section 68 of the extension tube 36, 38 within the channel 160 thereby securely attaching the extension tube 36, 38 to the lumen receptor 122. In order to allow for easy assembly (i.e., easy insertion of the section 68 of the extension tube 36, 38 into the channel 160), the channel 160 can be accessible via the top 156 of the support 152. For example, the channel 160 can extend inwardly from the top 156 of the support 152, such that the curved section 68 of the extension tube 36, 38 can be pressed into the channel 160 through the top 156 of the support 152.

FIG. 6B shows steps for attaching portions of the multi-lumen vascular access device 10 to the securement assembly 110 of FIGS. 6A and 6B. Initially, as in previous examples, the clinician attaches the bottom surface 118 of the base 116 of the adapter securement device 112 to a convenient location on skin and/or clothing of the patient proximate to a vascular access site. The clinician then inserts the adapter 30 of the multi-lumen vascular access device 10 into the housing or body 140 of the adapter receptor 138, such that the protrusions 144 or posts of the body 140 are inserted through the eyelets 64 of the adapter 30. The clinician then adheres the lumen securement device 114 to the patient at a convenient location proximate to but spaced apart from the adapter securement device 112. Once the lumen securement device 114 is adhered to the patient, the clinician inserts the curved sections 68 of the extension tubes 36, 38 into the channels 160 of the supports 152 of each of the lumen receptors 122 as shown in FIG. 6B. Once the curved sections 68 of the extension tubes 36, 38 are inserted into the channels 160, the clinician can rotate the supports 152, as shown by arrow Al (shown in FIG. 6A), to adjust the position of the connectors 26, 28 to improve accessibility of the connectors 26, 28.

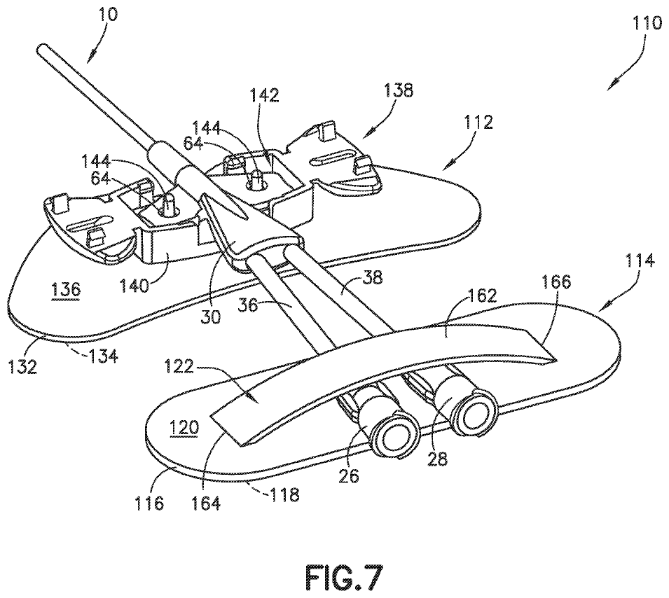

FIG. 7 shows another example of a securement assembly 110 for securing the multi-lumen vascular access device 10 to the patient. As in previous examples, the securement assembly 110 includes the adapter securement device 112 that secures the adapter 30 of the multi-lumen vascular access device 10 in place against skin or clothing of the patient proximate to a vascular access site. The securement assembly 110 also includes an example of a lumen securement device 114 for securing portions of the extension tubes 36, 38 of the multi-lumen vascular access device 10 in place against skin and/or clothing of the patient.

Unlike in previous examples, the lumen receptor 122 of the lumen securement device 114 of FIG. 7 is a simple strap 162 for securely attaching the connectors 26, 28 of the extension tubes 36, 38 to the base 116 of the lumen securement device 114. As shown in FIG. 7, the strap 162 includes ends 164, 166 connected to the base 116 for securing the multiple extension tubes 36, 38 to the base 116. In some examples, the ends 164, 166 of the strap 162 can be removable connected to the base 116 with a suitable reusable fastener. For example, the ends 164, 166 of the strap 162 can be connected to the base 116 by portions of a hook and loop fastener fabric (e.g., VELCRO®) on the strap 162 and on the base 116.

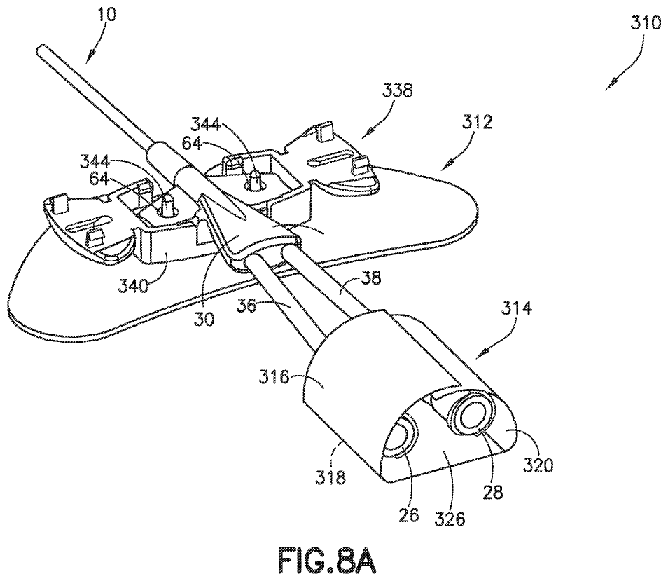

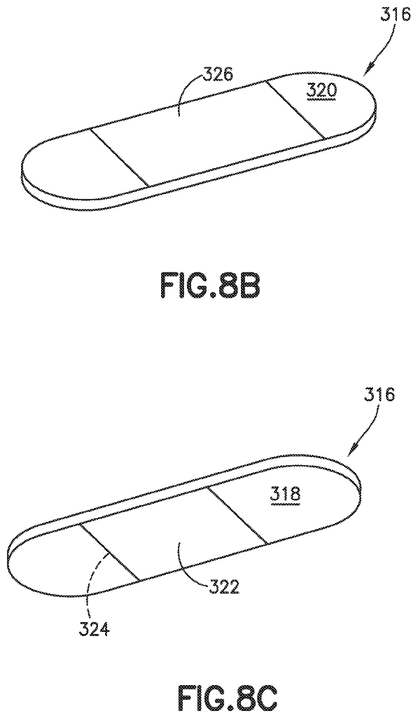

FIGS. 8A-8C show components of another example of a securement assembly 310 that can be used for attaching the multi-lumen vascular access device 10 to the patient. As in previous examples, the securement assembly includes an adapter securement device 312 that secures the adapter 30 of the multi-lumen vascular access device 10 in place against skin or clothing of the patient proximate to a vascular access site. The assembly also includes an example of a lumen securement device 314 for securing portions of the extension tubes 36, 38 of the multi-lumen vascular access device 10 in place against skin and/or clothing of the patient.

As shown in FIGS. 8A-8C, the lumen securement device 314 comprises one or more strips 316 of reusable tape. The strips 316 of reusable tape can include a first or bottom surface 318 with adhesive for securing a strip 316 of the tape to the skin or clothing of the patient. The strip 316 of tape can also include a second or top surface 320 configured to be folded around the connectors 26, 28 of the extension tubes 36, 38, as shown in FIG. 8A, for securing the extension tubes 36, 38 to the patient. In some examples, the strips 316 of reusable tape can be provided with a removable cover sheet 322 (shown in FIG. 8C) covering an adhesive portion 324 (shown in FIG. 8C), which can be removed in order to secure the strip 316 of tape to the patient.

In some examples, the tape can be dual-sided tape including one or more adhesive regions 326 (shown in FIGS. 8A and 8B) on the top surface 320 of the strip 316 of tape. For example, there can be an adhesive region 326 on a central portion of the top surface 320 of the strip 316 used for removably adhering one or more of the connectors 26, 28 to the strip 316 of tape. The strip 316 can also include adhesive regions 326 near ends of the top surface 320 for adhering the ends of the strip 316 together around the connectors 26, 28 as shown in FIG. 8A.

In order to secure the multi-lumen vascular access device 10 to the patient using the adapter securement device 312 and strip(s) 316 of re-usable tape of FIGS. 8A-8C, the clinician first attaches the adapter securement device 312 to a convenient location on skin and/or clothing of the patient proximate to a vascular access site, as in previous examples. The clinician then inserts the adapter 30 of the multi-lumen vascular access device 10 into a housing or body 340 of the adapter receptor 338, such that the protrusions 344 or posts of the body 340 are inserted through the eyelets 64 of the adapter 30. The clinician then removes the cover sheet 322 and adheres the bottom surface 318 of the strip 316 of re-usable tape to the patient at a convenient location proximate to but spaced apart from the adapter securement device 312. Once the strip 316 of tape is in place, the clinician places the connectors 26, 28 of the extension tubes 36, 38 on the top surface 320 of the strip 316. If present, the adhesive region 326 on the top surface 320 of the strip 316 can hold the connectors 26, 28 in place. The clinician then folds the ends of the strip 316 of tape over the connectors 26, 28, as shown in FIG. 8A. If present, adhesive regions 326 on ends of the strip 316 can be used for removably securing the strip 316 in a folded position.

As described in further detail herein, components of the securement assembly 110, 310 or securement device 210 can be provided as a kit of parts provided, for example, in a single package. In some examples, the kit can comprise the adaptor securement device 112, 312 and one or multiple strips 316 of the re-usable tape that have been cut to a desired length. Alternatively, the kit can include a roll of re-usable tape, allowing the clinician to cut one or more strips 316 of tape of desired lengths for attaching one or multiple extension tubes 36, 38 to the patient. In some examples, the multi-lumen vascular access device 10 can also be provided in the kit, along with the adapter securement device 312 and strips 316 of the removable tape.

FIGS. 9-11 show securement assemblies 110 that secure a multi-lumen vascular access 10 device to the skin and/or clothing of the patient. Unlike in previous examples, the multi-lumen vascular access device 10 includes additional extension tubes 70, 72, such as additional tubes and connectors of a catheter extension set, connected to extension tubes of the vascular access device. Specifically, as shown in FIGS. 9-11, the additional extension tubes 70, 72 include first or distal connectors 74, 76, such as male or female luer connectors, engaged to the corresponding connectors 26, 28 of the extension tubes 36, 28. The additional extension tubes 70, 72 also include proximal connectors 78, 80, such as male or female luer connectors, at a proximal end of the additional tubes 70, 72. The additional extension tubes 70, 72 also include lengths of flexible tubing connected between the distal connectors 74, 76 and the proximal connectors 78, 80.

As shown in FIG. 9, the securement assembly 110 comprises the adapter securement device 112 that secures the adapter 30 of the multi-lumen vascular access device 10 in place against skin or clothing of the patient proximate to a vascular access site, as in previous examples. The securement assembly 110 also includes a lumen securement device 114 for securing portions of the extension tubes 36, 76 and additional extension tubes 70, 72 of the multi-lumen vascular access device 10 in place against skin and/or clothing of the patient. The lumen securement device 114 includes the lumen receptors 122. The lumen receptors 122 in FIGS. 9-11 are configured to fixedly attached connectors 26, 28, 78, 80 to the lumen securement device 114 in a manner similar to the lumen securement device 114 shown in FIG. 4C. In other examples, the lumen securement device 114 of FIGS. 9-11 can be modified to include any of the other lumen receptors 112 described, such as the previously described lumen receptors 122 that allow the connectors 26, 28, 78, 80 to pivot, twist, or rotate relative to the base 116 of the lumen securement device 114.

As shown in FIG. 9, the lumen securement device 114, which can be similar or identical to the lumen securement device 112 shown in FIG. 4C, includes the base 116 comprising the bottom surface 118 comprising an adhesive configured for securing the base 116 to the patient and an opposing outwardly facing second or top surface 120. The lumen securement device 114 also includes multiple lumen receptors 122, each of which is configured to removably receive one of the extension tubes 36, 38 or additional tubes 70, 72 of the multi-lumen vascular access device 10 for fixedly connecting the extension tubes 36, 38 and additional tubes 70, 72 to the lumen securement device 114. For example, each lumen receptor 122 can include an arcuate receiving portion 150 sized to form a snap fit and fixed engagement with a narrow or distal end of one of the connectors 26, 28 of the extension tubes 36, 38 or with the proximal connectors 78, 80 of the additional extension tubes 70, 72.

As shown in FIG. 9, the lumen securement device 114 includes four lumen receptors 112 aligned along a virtual line V1 that is parallel with or substantially parallel (e.g., within about 10 degrees of parallel) with the longitudinal axis X1 of the base 116. The extension tubes 36, 38 extending from the adapter 30 are connected to middle or inner lumen receptors 122 with the connectors facing away from the adapter 30 (e.g., in a direction of arrow A2, as shown in FIG. 9). The additional extension tubes 70, 72 in FIG. 9 are connected to the outer lumen receptors 122 with the open proximal ends of the connectors 78, 80 facing towards the adapter 30, as shown by arrow A2 in FIG. 9. This configuration can be referred to as a “J-loop configuration” because the flexible tube of the additional extension tubes 70, 72 form a single curved portion that resembles the letter J.

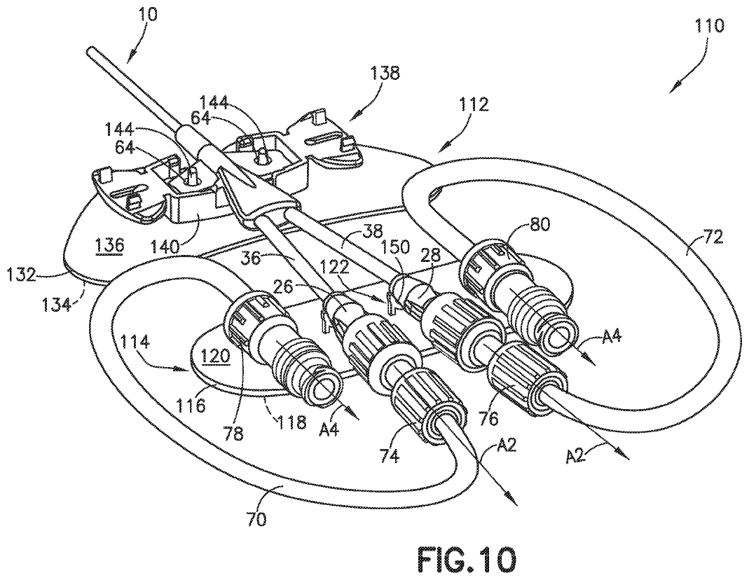

FIG. 10 shows the securement assembly 110 with the additional extension tubes 70, 72 in a “U-loop configuration” with the flexible tubes of the additional extension tubes 70, 72 forming two curved portions that resemble the letter U. More specifically, as shown in FIG. 10, the securement assembly 110 is provided for securing the multi-lumen vascular access device 10 in place against skin and/or clothing of the patient. As in previous examples, the adapter 30 of the vascular access device 10 is secured to the adapter securement device 112 and the connectors 26, 28 of the extension tubes 36, 38 are connected to the inner or middle lumen receptors 122 of the lumen securement device 114. As in FIG. 9, the connectors 26, 28 of the extension tubes 36, 38 are facing away from the adapter, as shown by arrow A2 in FIG. 10. As in FIG. 9, the proximal connectors 78, 80 of the additional extension tubes 70, 72 are connected to the outer lumen receptors 122 of the lumen securement device 114. However, in FIG. 10, the proximal connectors 78, 80 are facing away from the adapter (i.e., in a same direction as the connectors of the extension tubes), as shown by arrow A4 in FIG. 10. Further, the flexible tubes of the additional extension tubes 70, 72 are in the U-loop configuration forming a nearly complete (e.g., 360 degree) loop, as shown in FIG. 10.

In order to use the securement assembly of FIGS. 9 and 10 for securing the multi-lumen vascular access device 10 and additional extension tubes 70, 72 to the patient, the clinician first adheres the adapter securement device 112 to skin or clothing of the patient, as in previous examples. Once the adapter securement device 112 is in place, the clinician inserts the adapter 30 of the multi-lumen vascular access device 10 into the cavity 142 defined by the housing or body 140 of the adapter receptor 138 and positions the cover 146 over the cavity 142 to lock the adapter 30 in place. In a similar manner, the base 116 of the lumen securement device 114 is also adhered in place against the skin or clothing of the patient. Once the lumen securement device 114 is positioned at a desired location, the extension tubes 36, 38 are inserted into the middle lumen receptors 122 of the lumen securement device 114 as shown in FIGS. 9 and 10. For example, as previously described, the outer surface of the connectors 26, 28 can be pressed against the receiving portions 150 of the lumen receptors 122. Once the connectors 26, 28 of the extension tubes 36, 38 are in place, the clinician can attach the additional extension tubes 70, 72 to the extension tubes 36, 38 by, for example, inserting the distal connectors 74, 76 of the additional extension tubes 70, 72 into the connectors 26, 28 of the extension tubes 36, 38. In order to secure the additional extension tubes 70, 72 to the lumen securement device 114, the clinician then connects the proximal connectors 78, 80 of the additional extension tubes 70, 72 to the outer lumen receptors 122, as shown in FIGS. 9 and 10. If, as shown in FIG. 9, the proximal connectors 78, 80 of the additional extension tubes 70, 72 face towards the adaptor 30, then the additional extension tubes are in the J-loop configuration. If, as shown in FIG. 10, the proximal connectors 78, 80 are facing away from the adapter 30 (i.e., in the same direction as the connectors of the extension tubes) then the additional extension tubes are in a U-loop configuration.

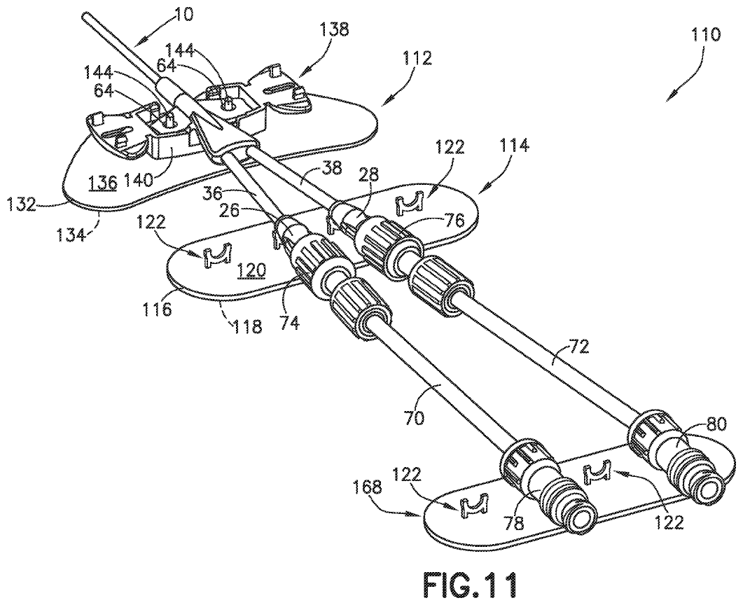

FIG. 11 shows another securement assembly 110 for securing the multi-lumen vascular access device 10 and additional extension tubes 70, 72 to the skin and/or clothing of the patient. As in the examples of FIGS. 9 and 10, the securement assembly of FIG. 11 comprises the adapter securement device 112 that secures the adapter 30 of the multi-lumen vascular access device 10 in place against skin or clothing of the patient proximate to a vascular access site, as in previous examples.

The securement assembly 110 also includes lumen securement devices, such as a first or distal lumen securement device 114 and a second or proximal lumen securement device 168, for securing portions of the extension tubes 36, 38 and the additional extension tubes 70, 72 in place against skin and/or clothing of the patient. The proximal and distal lumen securement devices 114, 168, which can be similar or identical to the lumen securement devices shown in FIGS. 4C, 9, and 10, include the base 116 comprising the bottom surface 118 comprising an adhesive configured for securing the base 116 to the patient and the opposing outwardly facing top surface 120. The lumen securement devices 114, 168 also includes multiple lumen receptors 122, each of which is configured to removably receive one of the extension tubes 36, 38 or additional extension tubes 70, 82 for fixedly connecting the extension tubes 36, 38 and additional extension tubes 70, 72 to the securement devices 114, 168. As shown in FIG. 11, the connectors 26, 28 of the extension tubes 36, 38 are connected to the lumen receptors 122 of the distal lumen securement device 112. The proximal connectors 78, 80 of the additional extension tubes 70, 72 are connector to lumen receptors 122 of the proximal lumen securement device 168.

In order to use the securement assembly 110 of FIG. 11 for securing the multi-lumen vascular access device 10 and additional extension tubes 70, 72 to the patient, the clinician first adheres the adapter securement device 112 to skin or clothing of the patient, as in previous examples. Once the adapter securement device 112 is in place, the clinician inserts the adapter 30 of the multi-lumen vascular access device 10 into the cavity 142 defined by the housing or body 140 of the adapter receptor 138 and can position the cover 146 over the cavity 142 to lock the adapter 30 in place. In a similar manner, the base 116 of the first or distal lumen securement device 114 is also adhered in place against the skin or clothing of the patient. Once the first or distal lumen securement device 112 is positioned at a desired location, the extension tubes 36, 38 are inserted into the middle lumen receptors 122 of the lumen securement device 114 as shown in FIG. 11. For example, as previously described, the outer surface of the connectors 26, 28 of the extension tubes 36, 38 can be pressed against the receiving portions 150 of the lumen receptors 122. Once the connectors 26, 28 of the extension tubes 36, 38 are in place, the clinician can attach the additional extension tubes 70, 72 to the extension tubes 36, 38 by, for example, inserting distal connectors 74, 76, such as male luer connectors, of the additional extension tubes 70, 72 into the connectors 26, 28 of the extension tubes 36, 38.

The clinician can also adhere the proximal lumen securement device 168 to the patient at a desired location. Once the proximal lumen securement device 168 is in place, the clinician can attach the proximal connectors 78, 80 of the additional extension tubes 70, 72 to the lumen receptors 122 of the proximal securement device 168, thereby securing the additional extension tubes 70, 72 to the securement assembly 110.

As previously described, any or all of the components of the multi-lumen vascular access devices 10 and securement assemblies 110, 310 or securement device 210 disclosed herein can be provided as a kit of parts for easy deployment and use by the clinician. For example, the kit can comprise the multi-lumen vascular access device 10, which includes the multi-lumen catheter 40, the adapter 30, and the multiple extension tubes 36, 38 extending from the proximal end 34 of the adapter 30. In some examples, the extension tubes 36, 38 can include the curved sections 68 configured to be received by the channels 160 of the cylindrical supports 152 of FIGS. 6A and 6B. The components of the multi-lumen vascular access device 10 can be provide in a disconnected configuration in the kit and can be assembled by the clinician immediately prior to use. The kit can also include the catheter extension set including the one or more additional extension tubes 70, 72 shown in FIGS. 9-11. As previously described, the additional extension tubes 70, 72 can include the distal connectors 74, 76, the proximal connectors 78, 80, and the flexible tubing extending between the distal connectors 74, 76 and the proximal connectors 78, 80.

The kits can also include one or more of any of the previously described lumen securement devices 114. In a simple example, the kit includes a single lumen securement device 114, such as a lumen securement device 114 including two lumen receptors 122 for securing the extension tubes 36, 38 of the vascular access device 10. In other examples, the kit can include multiple different types of lumen securement devices 114. For example, the kit can include lumen securement devices 114 with different numbers of lumen receptors 122, such as lumen securement devices 114 with two, three, four, or more receptors. The kit can also include lumen securement devices 114 with different types of lumen receptors 122. For example, the kit can include a lumen securement device 114 with lumen receptors 112 that allow connectors 26, 28 of the extension tubes 36, 38 to pivot. The kit can also include other lumen securement devices 114 with lumen receptors 122 that allow the connectors to rotate relative to the base 116 of the lumen securement device 114. The kit can include other lumen securement devices 114 with lumen receptors 122 that form a fixed engagement with the connectors 26, 28 of the extension tubes 36, 38.

The securement devices 210 and assemblies 110, 310 disclosed herein can be used with a variety of different types of vascular access devices and catheters available from numerous manufacturers and having a number of different designs and configurations. For example, as previously described, some multi-lumen vascular access devices include two extension tubes 36, 38 extending from the proximal end 34 of the adapter 30. In other examples, the vascular access device 10 can include three or more extension tubes extending from the adapter 30. Also, the adapter 30 or catheter hub can be provided in a variety of shapes, sizes, and configurations within the scope of the present disclosure.

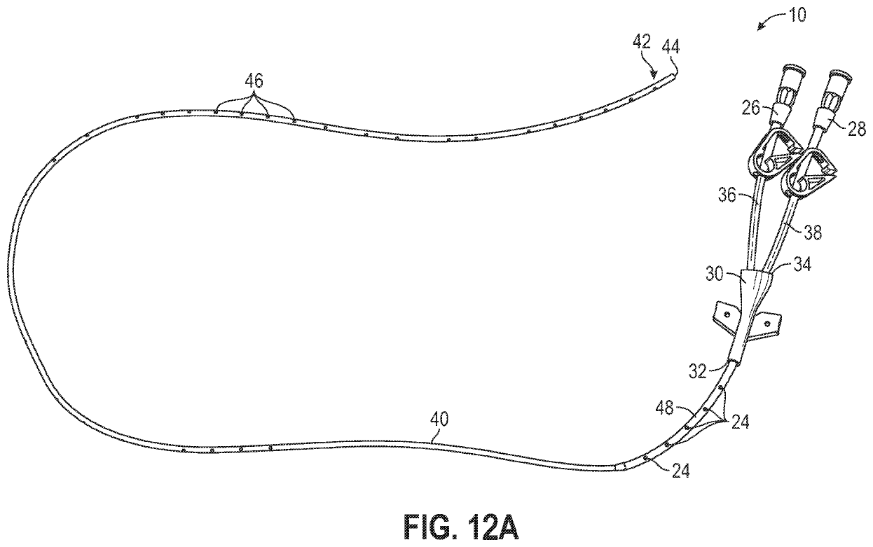

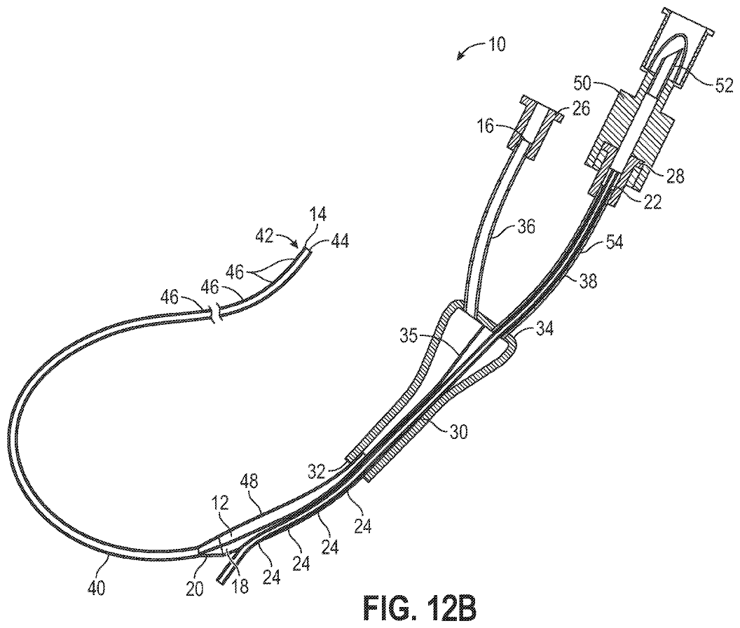

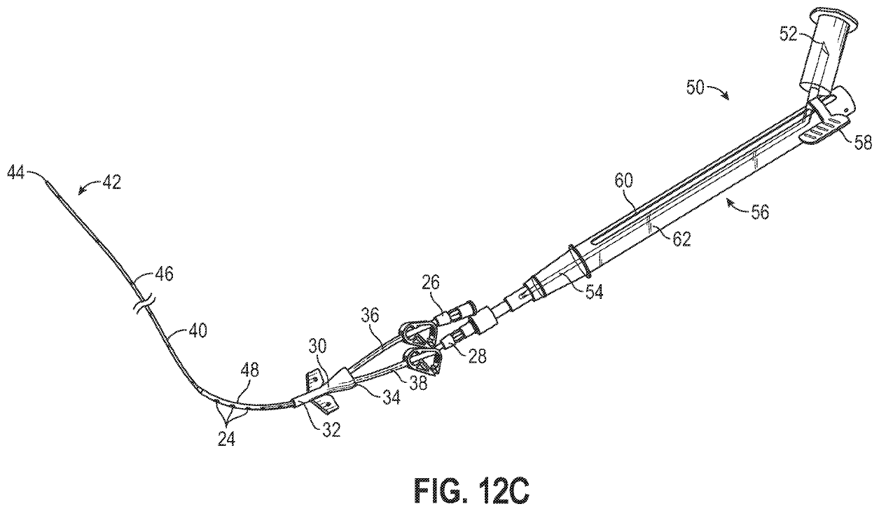

An exemplary multi-lumen vascular access device 10 that can be used with the securement devices 210 and assemblies 110, 310 of the present disclosure is shown in FIGS. 12A-12C. However, other configurations and arrangements of vascular access devices 10 for use with the securement devices 210 and assemblies 110, 310 disclosed herein will also be apparent to those skilled in the art.

As shown in FIGS. 12A and 12B, the vascular access device 10 includes a first lumen 12, which comprises a distal end 14 and a proximal end 16. The vascular access device 10 can also include a second lumen 18, which may include a distal end 20 connected to a catheter 40 and a proximal end 22. The first and second lumens 12, 18 extend through a catheter hub or adapter 30 as shown in FIG. 12B.

In some examples, the distal end 20 of the second lumen 18 can be proximal to the distal end 14 of the first lumen 12. In some examples, the second lumen 18 terminates closer to the distal end 14 of the first lumen 12 when the catheter 40 is a midline catheter as opposed to a peripherally-inserted central catheter. The vascular access device 10 may include an opening 24 or a valve, which may be adjacent to the distal end 20 of the second lumen 18. In some examples, the vascular access device 10 may include multiple openings 24 or multiple valves, which may be disposed along the second lumen 18.

The vascular access device 10 can also include a first connector 26, such as a male or female luer connector, disposed at the proximal end 16 of the first lumen 12, and a first extension tube 36 extending from the catheter adapter 30 to the first connector 26. The vascular access device 10 can also include a second connector 28, such as a male or female connector, disposed at the proximal end 22 of the second lumen 18 and a second extension tube 38 extending from the catheter adapter 30 to the second connector 28. In some examples, a needleless access connector (not illustrated) can be coupled to the first connector 26 and/or the second connector 28. Optionally, the first connector 26 can include color and/or markings different from the second connector 28, which may facilitate differentiation of the first lumen 12 and the second lumen 18 by the clinician. In a similar manner, the first extension tube 36 may include color and/or markings different from the second extension tube 38, which may facilitate differentiation of the first lumen 12 and the second lumen 18 by the clinician. In some examples, the needleless access connector may include an anti-reflux or positive displacement connector.

The vascular access device 10 also includes the catheter adapter 30, which can include a distal end 32 and a proximal end 34. The vascular access device 10 can also include a divider 35, which may extend between the distal end 32 of the catheter adapter 30 and the proximal end 34 of the catheter adapter 30. The divider 35 can separate the first lumen 12 and the second lumen 18 within the catheter adapter 30. The first lumen 12 and the second lumen 18 may be side by side, as illustrated, for example, in FIG. 12B.

The vascular access device 10 also includes the first extension tube 36, which extends from the proximal end 34 of the catheter adapter 30 to the first connector 26, and the second extension tube 38, which extends from the proximal end 34 of the catheter adapter 30 to the second connector 28. The vascular access device 10 also includes the catheter 40 extending from the proximal end 34 of the catheter adapter 30. The catheter 40 can be a peripheral intravenous catheter, a peripherally-inserted central catheter, or a midline catheter. In some examples, a distal end 42 of the catheter 40 may include a distal opening 44 and/or one or more valves or one or more side holes 46. In some examples, a proximal end of the catheter 40 may be secured within the catheter adapter 30. The first extension tube 36 and an entire length of the catheter 40 may include the first lumen 12. In some examples, the second extension tube 38 and a proximal portion 48 of the catheter 40 may include the second lumen 18. Also, the catheter 40 can include a portion of the divider 35, which can extend through the proximal portion 48 of the catheter 40. In some examples, the first lumen 12 and the second lumen 18 are side by side within the catheter adapter 30 and the proximal portion 48.

In some examples, the first extension tube 36 can include a clamp (shown in FIG. 12A), which may prevent blood from flowing through the first lumen 12 in response to the clamp being in a closed position. The second extension tube 38 can also include a clamp (shown in FIG. 12A), which may prevent blood from flowing through the second lumen 18 in response to the clamp being in a closed position. In some examples, the clamp on the first extension tube 36 may include color and/or markings different from the second extension tube 38 and/or the clamp on the second extension tube 38, which may facilitate differentiation of the first lumen 12 and the second lumen 18 by the clinician.

As shown in FIGS. 12B and 12C, in some examples, the vascular access device 10 includes a blood collection device 50, which may be coupled to the second connector 28 and/or in fluid communication with the second lumen 18. The blood collection device 50 may be directly coupled to the second connector 28 or coupled to the second connector 28 via a needleless connector. The blood collection device 50 may include an instrument 54 extending through the second lumen 18 and out the opening 24 or the valve. The instrument 54 can include a tube, a guidewire, a wire, an obturator, a probe, a sensor, or any other suitable instrument. In some examples, the multiple openings 24 or the multiple valves may be adjacent to the distal end 20 of the second lumen 18.

The blood collection device 50 can include or correspond to a blood collection container. The blood collection container can include a syringe, an evacuated blood collection tube, a small sample collection device, or any other container. In some examples, the blood collection device 50 may include VACUTAINER® or a VACUTAINER® LUER-LOK™, available from Becton, Dickinson and Company of Franklin Lakes, N.J., which may be configured to receive the blood collection container. In some examples, the blood collection device 50 may include a needle 52, which can be disposed within an elastomeric sheath, and which may be configured to insert into the blood collection container. In response to the blood collection container pushing the elastomeric sheath distally, the needle 52 may pierce the elastomeric sheath and be inserted into the blood collection container.

As shown in FIG. 12C, the blood collection device 50 may also include a delivery device 56. In some examples, the instrument 54 may be delivered through the second lumen 18 and out the opening 24 or the valve via the delivery device 56. The delivery device 56 can include an advancement element 58. The advancement element 58 can include a tab or a grip, which may be moved by the clinician to advance the instrument 54 in a distal direction and/or retract the instrument 54 in a proximal direction. The advancement element 58 can be coupled to instrument 54. Specifically, the advancement element 58 can be slidable along a slot 60 disposed within a housing 62.

The delivery device 56 can include any suitable delivery device known in the art configured to be connected to a medial connector, such as a male or female luer connector. Exemplary delivery devices that can be used with the multi-lumen vascular access device 10 and securement devices of the present disclosure are described, for example, in the following patents and published patent applications: U.S. Pat. No. 11,406,679, entitled “Delivery device for a vascular access instrument”; U.S. Pat. No. 11,547,832, entitled “Catheter delivery device and related systems and methods”; and U.S. Patent Application Publication No. 2019/0021640, entitled “Extension housing a probe or intravenous catheter”, which are incorporated herein by reference in their entireties.

While examples of the securement devices, securement assemblies, kits and methods of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is

1. A securement device configured to be connected with multiple extension tubes extending from an adapter of a multi-lumen access device, the securement device comprising:
at least one base comprising a first surface comprising an adhesive configured for securing the at least one base to a patient and an opposing outwardly facing second surface; and
at least one lumen receptor connected to and extending from the second surface of the at least one base,
wherein the at least one lumen receptor is configured to removably receive one or more of the multiple extension tubes of the multi-lumen access device for securing the multiple extension tubes of the multi-lumen access device to the at least one base.

2. The securement device of claim 1, wherein the at least one lumen receptor comprises a plurality of lumen receptors, each of which is configured to removably receive one of the multiple extension tubes of the multi-lumen access device for securing the multiple extension tubes to the at least one base.

3. The securement device of claim 1, wherein the at least one lumen receptor comprises a first support and a second support connected to the second surface of the at least one base and a cradle connected between the first support and the second support configured to removably receive a connector of one of the extension tubes of the multi-lumen access device.

4. The securement device of claim 3, wherein the cradle is sized to provide a removable snap fit engagement with an outer surface of the luer connector of the extension tube of the multi-lumen access device.

5. The securement device of claim 3, wherein the cradle is pivotally connected to the first support and the second support.

6. The securement device of claim 1, further comprising an adapter receptor connected to the second surface of the at least one base configured to removably engage the adapter of the multi-lumen access device.

7. The securement device of claim 6, wherein the adapter receptor comprises a body defining a cavity sized to receive the adapter, a plurality of protrusions sized to be inserted through corresponding eyelets of the adapter, and a cover configured to be positioned over the protrusions and/or adapter for removably securing the adapter to the adapter receptor.

8. The securement device of claim 6, wherein the at least one base comprises an adapter securement portion connected to the adapter receptor, a lumen securement portion connected to the plurality of lumen receptors, and an intermediate portion between the adapter securement portion and the lumen securement portion.

9. The securement device of claim 1, wherein the at least one lumen receptor comprises a support comprising a bottom connected to the at least one base, a top opposite the bottom, and an annular side surface extending between the top and the bottom, the support being rotatably connected to the at least one base and defining a channel sized to receive a section of one of the extension tubes of multi-lumen access device.

10. The securement device of claim 1, wherein the at least one lumen receptor comprise depressions or holes sized to receive corresponding pins extending from a connector of one of the extension tubes of the multi-lumen access device for pivotally connecting the connector to the at least one lumen receptor.

11. The securement device of claim 1, wherein the at least one lumen receptor comprises four lumen receptors, with two of the lumen receptors configured to receive the multiple extension tubes and two of the lumen receptors configured to receive additional extension tubes of an extension set, wherein the four lumen receptors are aligned along a virtual line that is parallel with or substantially parallel with a longitudinal axis of the at least one base.

12. A securement assembly for securement of multiple extension tubes extending from an adapter of a multi-lumen access device, the securement assembly comprising:
at least one adapter securement device comprising an adhesive base and an adapter receptor connected to the base configured to removably engage the adapter of the multi-lumen access device; and
at least one lumen securement device comprising an adhesive base configured to removably receive the multiple extension tubes of the multi-lumen access device for securing the multiple extension tubes of the multi-lumen access device to the adhesive base of the at least one lumen securement device.

13. The securement assembly of claim 12, wherein the lumen securement device comprises a plurality of lumen receptors, each of which is configured to removably receive one of the multiple extension tubes of the multi-lumen access device for securing the multiple extension tubes to the adhesive base of the lumen securement device.

14. The securement assembly of claim 12, wherein the at least one lumen securement device comprises one or more strips of reusable tape.

15. The securement assembly of claim 12, further comprising the multi-lumen access device comprising the adapter and the multiple extension tubes extending from the adapter and comprising connectors at proximal ends thereof.

16. The securement assembly of claim 15, further comprising at least one additional extension tube comprising a distal end connected to the connector of one of the multiple extension tubes and a proximal end connected to a lumen receptor of the at least one lumen securement device.

17. The securement assembly of claim 15, wherein one or more of the multiple extension tubes of the multi-lumen access device are pivotally or rotatably engaged to the at least one lumen securement device.

18. A kit for securing a multi-lumen access device to a patient, comprising:
a multi-lumen access device comprising a multi-lumen catheter comprising a distal end configured for insertion to vasculature of a patient, an adapter connected to a proximal end of the multi-lumen catheter, multiple extension tubes extending proximally from the adapter, and connectors at proximal ends of the extension tubes;
at least one adapter securement device comprising an adhesive base and an adapter receptor connected to the base configured to removably engage the adapter of the multi-lumen access device; and
at least one lumen securement device comprising an adhesive base, wherein the at least one lumen securement device is configured to removably receive the multiple extension tubes of the multi-lumen access device for securing the extension tubes of the multi-lumen access device to the adhesive base of the at least one lumen securement device.

19. The kit of claim 18, wherein the at least one lumen securement device comprises reusable tape.

20. The kit of claim 18, further comprising an extension set comprising at least one additional extension tube configured to be connected to one of the connectors on the proximal end of the extension tube,
wherein the at least one lumen securement device comprises a plurality of lumen receptors, wherein at least two of the plurality of lumen receptors are configured to engage the connectors on the proximal ends of the extension tubes and at least two of the plurality of lumen receptors are configured to engage proximal connectors of the at least one additional extension tube of the extension set.
